SEQUENCE LISTING (8 sequences)

SEQ ID NO: 1 - DNA, 20 bp, Synthetic construct
  source 1..20: mol_type = other DNA
cacagaactaaaagtccggg

SEQ ID NO: 2 - DNA, 20 bp, Synthetic construct
  source 1..20: mol_type = other DNA
cgcattgcgaatgcgctcgt

SEQ ID NO: 3 - RNA, 20 nt, Synthetic construct
  source 1..20: mol_type = other RNA
aaatataactcaggaccccg

SEQ ID NO: 4 - RNA, 20 nt, Synthetic construct
  source 1..20: mol_type = other RNA
taggatatggaaccaaactg

SEQ ID NO: 5 - RNA, 20 nt, Synthetic construct
  source 1..20: mol_type = other RNA
agtgagtcgcagtttagcgt

SEQ ID NO: 6 - RNA, 20 nt, Synthetic construct
  source 1..20: mol_type = other RNA
gaagttaggactcatcacag

SEQ ID NO: 7 - RNA, 20 nt, Synthetic construct
  source 1..20: mol_type = other RNA
cgacgctcaacaccaaccgg

SEQ ID NO: 8 - RNA, 20 nt, Synthetic construct
  source 1..20: mol_type = other RNA
acgcggaacaagatgcagag

(19) United States
(12) Patent Application Publication
(10) Pub. No.: US 2025/0082649 A1
(43) Pub. Date: Mar. 13, 2025

(54) ALTERATION OF NUTRIENT PREFERENCES VIA MODULATION OF NUTRIENT RECEPTORS IN THE GUT

(71) Applicant: The Trustees of Columbia University in the City of New York, New York, NY (US)

(72) Inventors: Mengtong Li, New York, NY (US); Charles S. Zuker, New York, NY (US); Zhengyuan Lu, New York, NY (US)

(21) Appl. No.: 18/814,140

(22) Filed: Aug. 23, 2024

Related U.S. Application Data
(63) Continuation of application No. PCT/US2023/063286, filed on Feb. 24, 2023.
(60) Provisional application No. 63/314,616, filed on Feb. 28, 2022.
(60) Provisional application No. 63/313,960, filed on Feb. 25, 2022.

(51) Int. Cl. A61K 31/5513; A61K 31/47; A61K 31/662; A61K 38/22; A61P 3/02

(52) U.S. Cl. CPC A61K 31/5513; A61K 31/47; A61K 31/662; A61K 38/2207; A61P 3/02

(57) ABSTRACT

Provided herein are methods of regulating a subject's preference for fat and/or sugar and methods of screening a substance for an ability to activate a nutrient receptor in a subject's gut.

### CROSS-REFERENCE TO RELATED APPLICATIONS

This application is a continuation of International Application Serial Number PCT/US2023/063286, filed on Feb. 23, 2023, which claims priority to U.S. Provisional Application Ser. No. 63/313,960, filed on Feb. 25, 2022, and U.S. Provisional Application Ser. No. 63/314,616, filed on Feb. 28, 2022, the disclosures of which are hereby incorporated by reference in their entireties.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: 8,152 bytes; XML file named “COLUM_42431_303_SequenceListing” created on Nov. 20, 2024.

### TECHNICAL FIELD

Provided herein are methods of regulating a subject's preference for fat and/or sugar, as well as methods of screening a substance for an ability to activate a nutrient receptor in a subject's gut.

### BACKGROUND

Developed and underdeveloped countries have experienced catastrophic increases in the consumption of processed foods high in sugar and fat. These changes in dietary intake have been implicated in malnutrition, including over- and undernutrition, and have been linked to a wide number of metabolic disorders and related comorbidities.

Sugar and fat are essential nutrients, and consequently, animals have evolved taste signaling pathways that detect and respond to sweet and fat stimuli, leading to appetitive and consummatory behavior. Yet, the development of nutrient preference is mediated by the gut brain-axis, rather than the immediate responses triggered by the taste system.

### SUMMARY

Disclosed herein are methods of regulating a subject's preference for fat and/or sugar comprising administering to the subject a molecule that activates or inhibits one or more of a GPR40 receptor, a GPR120 receptor, and a CCK signaling pathway.

Also disclosed herein are methods of screening a substance for an ability to activate a nutrient receptor in a subject's gut, the method comprising administering a substance to a subject wherein the subject's vagal sensory neurons express a genetically encoded calcium indicator (GECI); imaging the vagal sensory neurons of the subject; and detecting a presence or absence of a signal from the GECI, wherein the presence of the signal in a region of the subject's vagal sensory neurons that are responsive to nutrient receptor activation in the gut indicates that the substance activates the nutrient receptor.

### BRIEF DESCRIPTION OF THE DRAWINGS

The summary, as well as the following detailed description, is further understood when read in conjunction with the appended drawings. For the purpose of illustrating the methods, there are shown in the drawings exemplary embodiments of the methods; however, the methods are not limited to the specific embodiments. In addition, the drawings are not necessarily drawn to scale. In the drawings:

FIG. 1A is a graph illustrating the cumulative number of licks of a bottle containing a fat emulsion (1.5% Intralipid, IL) or an artificial sweetener (3 mM AceK) over a 48 hour session. The color bars at the top show lick rasters for fat (left) and sweet (right) from the first and last 2000 licks of the behavioral test. Note that by 24 h the animals begin to drink almost exclusively from the fat bottle. FIG. 1B depicts preference plots for fat versus sweet. In these experiments, mice began the behavioral preference test preferring sweet (preference index<0.5), but in all cases they switched their preference to fat (n=7 mice, two-tailed paired t-test, P=1.9×10⁻⁵). FIG. 1C depicts a schematic of fat stimulation of Fos induction. Strong Fos labelling was observed in the cNST (outlined) upon ingestion of Intralipid (IL, 20%) but not control stimuli (0.3% xanthan gum). Scale bars, 100 μm. FIG. 1D shows the quantification of Fos-positive neurons. The equivalent area of the cNST (200 μm×200 μm; Bregma—7.5 mm) was processed, and positive neurons were counted for the different stimuli. Two-sided Mann-Whitney U-test between Xanthan Gum (XG) and Intralipid (IL) (n=5 mice), P=0.0079. Values are mean±s.e.m. FIG. 1E illustrates that the direct intragastric infusion of fat (IL) also robustly activates the cNST. Scale bars, 100 μm. PBS was used as a control stimulus to maintain osmolarity between experimental and control infusion. Note strong labelling in the cNST. FIG. 1F provides the quantification of Fos-positive neurons. Two-sided Mann-Whitney U-test between PBS control (n=5 mice) and Intralipid (n=5 mice), P=0.008. Values are mean±s.e.m.

FIG. 2A depicts a graph illustrating that fat preference is mediated by the gut-brain axis. The left panel is a schematic of the silencing strategy. TRAP2 mice were stimulated with Intralipid (20%) to induce expression of Cre recombinase in the cNST. An AAV-DIO-TetTox virus was then targeted bilaterally to the cNST for silencing. The right panel is a bar graph that shows consumption over the 48 hr preference test. Control animals developed a strong preference for Intralipid versus sweetener. In contrast, animals in which fat-activated cNST neurons were silenced no longer exhibit a preference for fat over sweetener. Two-sided Mann-Whitney U-test between control (n=10 mice) and fat-TetTox (n=9 mice) fat consumption, P=0.0014. Data are mean±s.e.m. FIG. 2B Depicts fibre photometry used to monitor activity in the cNST in response to intestinal delivery of fat. Excitatory neurons in the cNST were targeted with GCaMP6s using Vglut2-Cre mice. FIG. 2C illustrates neural responses following intestinal delivery of fat (10% linoleic acid) or sugar (500 mM glucose) as a control. The solid trace is the average and the shaded area represents s.e.m. The responses after bilateral vagotomy are shown. n=6 mice. NR, normalized response. FIG. 2D shows the quantification of neural responses before and after vagotomy. Two-tailed paired t-test, P=4.6×10⁻⁸ (sugar), P=4.9×10⁻⁸ (fat). Data are mean±s.e.m. Note total loss of responses following bilateral vagotomy. FIG. 2E provides imaging of calcium responses in vagal sensory neurons while simultaneously delivering stimuli into the intestines. Heat maps depict z-score-normalized fluorescence traces from vagal neurons identified as fat responders (n=84 neurons from 515 cells from 8 ganglia). Each row represents the average activity of a single cell to four trials. Stimulus window (10 s) is shown by dotted white lines. Note strong responses to intestinal delivery of fat but not to control stimuli. Vehicle control, 0.1% xanthan gum plus 0.05% Tween 80; fat, 10% linoleic acid (LA) emulsified in vehicle control solution. FIG. 2F depicts sample traces of vagal neuron responses to alternating 10 s pulses of control (XG dotted lines) and fat stimuli (LA dotted lines). Note the reliability and robustness of responses to fat. FIG. 2G displays heat maps summarizing responses to interleaved 10 s stimuli of fat (10% linoleic acid, LA) and sugar (500 mM glucose, Glu). Each row represents the average activity of a single cell during three exposures to the stimulus. Stimulus window is indicated by the dotted white lines. Shown in the upper panels are 151 neurons that responded to intestinal application of both fat and glucose (“nutrient responders”). The bottom panel shows a different pool of 153 neurons that responded only to fat; no responses to intestinal sugar were detected in the “fat-only responders” (bottom panels). n=22 vagal ganglia, 1813 neurons were imaged.

FIG. 3A illustrates imaging of calcium responses in vagal sensory neurons in Vglut2-Cre; Ai96 animals while delivering fat, sugar, or amino acid stimuli into the intestines (10% linoleic acid, 500 mM glucose or a 250 mM amino acid mixture). Heat maps depict z-score-normalized fluorescence traces of nutrient-responders (top) and fat-only responders (bottom) from 641 neurons of 8 mice. Each row represents the average activity of a single cell to two trials. Stimulus window (10 s for fat or sugar, 60 s for amino acids) is shown by dashed white lines. FIG. 3B depicts calcium responded following inhibition of CCK signaling with devazepide (4 mg/kg, 200 μL), a CCKAR antagonist. Note that blocking CCKAR receptor activation dramatically abolishes fat-, sugar- and amino acid-evoked activity in nearly all the nutrient responders (compare POST vs PRE, top panels). By contrast, the CCKAR blocker had no effect on the fat-evoked activity in the fat-only responders (compare POST vs PRE, bottom panels). FIG. 3C displays a cartoon of the gut-to-brain nutrient sensing vagal axis. The bottom inset illustrates CCK-expressing enteroendocrine cells (EEC) in the intestines. The top inset is a two-dimensional t-distributed stochastic neighbor embedding (tSNE) plot of the transcriptome of mouse vagal nodose neurons. Shown are the clusters of vagal neurons expressing the CCK-A Receptor (CCKAR) FIG. 3D illustrates that CCKAR-expressing neurons respond to intestinal stimulation with nutrients. An engineered CCKAR-iCre was used to drive GCaMP6s expression in CCKAR vagal neurons (Ai162). 724 imaged neurons were analyzed from 12 ganglia. Shown are heat maps depicting z-score-normalized fluorescence traces of the CCKAR-positive neurons to intestinal delivery of fat (10% linoleic acid), sugar (500 mM glucose) or amino acids (250 mM amino acid mixture).

FIG. 4A illustrates a tSNE plot of the transcriptome of vagal nodose neurons. While CCKAR-positive neurons comprise nutrient responders and a small fraction of the fat-only responders, a subset of CCKAR neurons marked by VIP expression (circled cluster in the cartoon) define the nutrient responders. FIG. 4B depicts the calcium responses recorded in vagal ganglia of animals with GCaMP6s expressed in VIP neurons while infusing fat, sugar or amino acid stimuli into the intestines. Heat maps show z-score-normalized fluorescence traces. Approximately 30% of VIP vagal neurons responded to nutrient stimuli (n=60/203 neurons from 9 ganglia). Stimuli: 10% linoleic acid, 500 mM glucose, 250 mM amino acid mixture. FIG. 4C shows the strategy for silencing of VIP neurons in the vagal ganglia by bilateral injection of AAV-DIO-TetTox into the nodose in VIP-Cre animals. FIG. 4D & FIG. 4E illustrate fat and sugar preference tests for wild type mice (open bars) and mice with silenced VIP-expressing vagal neurons (VIPTx, shaded bars). Wild type mice develop strong preference for fat after a standard 48 hr fat vs. sweetener choice test (n=10). In contrast, silencing of VIP vagal neurons abolishes the development of fat preference (n=13 VIPTx mice). Two-sided Mann-Whitney U-test, WT vs. VIPTx fat consumption, P=6.2×10⁻⁵. Values are mean±s.e.m. Silencing of VIP vagal neurons abolishes the development of sugar preference, compare wildtype (n=10) versus silenced animals (n=13). Two-sided Mann-Whitney U-test, WT vs. VIPTx sugar consumption, P=0.05. Values are mean±s.e.m. Note that since sugar and the artificial sweetener trigger strong taste responses, animals with silenced gut-brain signals end up consuming almost as much sweetener as sugar over the 48 hr test. FIG. 4F shows the strategy for chemogenetic activation of VIP vagal neurons. An excitatory DREADD receptor (via AAV-DIO-hM3Dq) was targeted bilaterally to the nodose of VIP-Cre mice. The mice were then tested for their basal preference to cherry and grape flavor for 48 hrs (Pre). Mice were conditioned and tested using the less-preferred flavor plus the DREADD agonist clozapine (Post). FIG. 4G. demonstrates that wild-type mice presented with clozapine (1 mg/kg) in the less preferred flavor do not switch their preference, and maintain their basal, original flavor choice (left panel). Pre, 40.6±4.6%; Post, 34.3±4.5%; n=8 mice; two-sided Mann-Whitney U-test, P=0.061. Preference Index values are mean±s.e.m. After associating clozapine-mediated activation VIP vagal neurons with the less-preferred flavor, all the animals expressing DREADD in VIP vagal neurons switched their preference (right panel) (n=6 mice; two-sided Mann-Whitney U-test, P=9.6×10⁻⁴).

FIG. 5A displays a single cell RNA seq atlas of nodose ganglia, showing vagal clusters for VIP, TrpA1, GPR65, calca, Oxtr, and Piezo2. FIG. 5B illustrates that the vagal cluster expressing TRPA1 (Trpa1-Cre; Ai162) responded selectively to intestinal delivery of fat, but not sugar or amino acid stimuli. The heat maps show z-score-normalized fluorescence traces. Of 163 imaged neurons from 5 ganglia, ˜24% responded to fat. Stimuli: 10% linoleic acid, 500 mM glucose, 250 mM amino acid mixture. FIG. 5C shows the strategy for silencing of TRPA1 neurons in the vagal ganglia by bilateral injection of AAV-DIO-TetTox into the nodose in Trpa1-Cre animals. FIG. 5D illustrates the fat and sugar preference tests on control mice (left) and mice with silenced TRPA1-expressing vagal neurons (Trpa1-Tx) (right). Control mice develop strong preference for fat and sugar after 48 h (n=7). By contrast, silencing of TRPA1 vagal neurons abolishes the development of fat but not sugar preference (n=6, right). Two-sided Mann-Whitney U-test, control versus Trpa1-Tx for sugar, P=0.23; control versus Trpa1-Tx for fat, P=1.1×10⁻³. Data are mean±s.e.m.

FIG. 6A depicts the experimental paradigm in which knockout mice were engineered for 3 candidate gut fat receptors, and generated all potential knockout combinations. Vagal responses were then recorded to intestinal delivery of fat (10% linoleic acid) and sugar (500 mM glucose) stimuli, and separately tested the knockout mice behaviorally for the development of fat and sugar preference. FIG. 6B illustrates heat maps that depict z-score-normalized fluorescence traces from vagal neurons in response to intestinal fat and sugar delivery. SGLT1 functions as the gut-to-brain sugar receptor and none of the imaged neurons responded to sugar in the SGLT1 knockout animals. However, responses to fat are unaffected mice (n=174 responders from 903 imaged neurons; n=10 ganglia). FIG. 6C displays the quantifications of vagal neurons responding to intestinal delivery of fat (10% LA) in control and the various receptor knockout combinations. Vagal responses were dramatically impacted only in the GPR40/GPR120 double knockout and in the triple knockout animals. ANOVA with Tukey's honestly significant difference (HSD) to WT. CD36 KO (n=6 mice), P=0.99; GPR40 KO (R40, n=7 mice), P=0.89; GPR120 KO (R120, n=6 mice), P=0.53; CD36/GPR40 double KO (CD36/R40, n=6 mice), P=0.96; CD36/GPR120 double KO, (CD36/R120, n=8 mice), P=0.99; GPR40/GPR120 double KO (R40/120, n=7 mice), P=5×10⁻⁶; CD36/GPR40/GPR120 triple KO (3KO, n=6 mice), P=4×10⁻⁶. Values are mean±s.e.m. FIG. 6D shows sample heat maps illustrating the selective loss of fat responses in the GPR40/GPR120 double knockout and the triple knockout. Note normal responses to intestinal delivery of sugar both in the double (n=51 from 428 imaged neurons of 6 ganglia), and the triple fat receptor knockout animals (n=44 out of 326 imaged neurons from 6 ganglia). FIG. 6E illustrates wild type and knockout animals tested for the development of fat preference. GPR40/GPR120 double knockouts (R40/120, n=7 mice, P=0.81) and CD36/GPR40/GPR120 triple knockouts (3KO, n=9 mice, P=0.46) mice fail to develop a preference for fat. Left bars: initial preference; Right bars: preference at the end of the 48 hr test. All other combinations of knockouts are capable of developing robust behavioral preference for fat, just like wild type control mice. CD36 KO, (n=8 mice), P=0.0048; GPR40 KO, (R40, n=12 mice), P=1.0×10⁻⁴; GPR120 KO, (R120, n=14 mice), P=1.0×10⁻⁴; CD36/GPR40 KO, (D36/R40, n=5 mice), P=0.02; CD36/GPR120 KO (D36/R120, n=6 mice), P=0.0017; wild type mice (WT, n=11 mice), P=2.0×10⁻⁶. All statistics are from two-tailed paired t-tests evaluating Pre versus Post preference. Values are mean±s.e.m. FIG. 6F demonstrates that knockout mice were unable to develop fat preference exhibit perfectly normal preference for sugar stimuli. Wild type (WT, n=10 mice), P=2.9×10⁻⁵; GPR40^{-/-}GPR120^{-/-} (R40/120, n=9 mice), P=8.0×10⁻⁵; CD36^{-/-}GPR40^{-/-}GPR120^{-/-} (3KO, n=7 mice), P=1.9×10⁻³. All statistics are from two-tailed paired t-tests evaluating Pre versus Post preference. Values are mean±s.e.m.

FIG. 7A and FIG. 7B show the immediate attraction to sweet and fat. Graphs of lick counts from brief-access (30 min) two-bottle tests. In FIG. 7A, artificial sweetener (3 mM AceK) versus water, n=9 mice, two-tailed paired t-test, P=2×10⁻⁶. In FIG. 7B, fat (1.5% Intralipid, IL) versus water, n=9 mice, two-tailed paired t-test, P=2×10⁻⁵. Values are mean±s.e.m. Note strong innate attraction to sweet and fat stimuli. FIG. 7C shows that immediate attraction to fat is abolished in TRPM5 knock-out animals. Shown are results from 30 min two-bottle test of fat (1.5% Intralipid, IL) versus water in wild type mice (left panel, n=11 mice) versus TRPM5 knockout mice (right panel, n=12 mice). TRPM5 knock-out animals are blind to the taste of fat. Two-sided Mann-Whitney U-test wild type versus TRPM5 knockout fat consumption: P=1.6×10⁻³. Note that there is no innate attraction to either bottle, with the animals randomly choosing to consume from either one. FIG. 7D illustrates that in a 48 h two-bottle fat preference test, TRPM5 KO animals still developed strong post-ingestive preference to fat (n=6 mice, two-tailed paired t-test, P=7.4×10⁻⁴). FIG. 7E demonstrates that development of fat preference is independent of caloric content. To test the effect of calories, preference between a caloric sugar (fructose) versus fat was examined. Importantly, a sugar was used (fructose) that does not activate SGLT1, and therefore does not trigger post-ingestive preference, thus the effect of calories without the confound of having two preference-triggering stimuli can be isolated (i.e., glucose versus fat). Cartoon on the top illustrates the behavioral arena; mice were allowed to choose between fructose (0.15 kcal/ml) and fat (IL at 0.15 kcal/ml). Paired t-test, P=8×10⁻⁴, n=7. FIG. 7F illustrates a test similar to FIG. 7E, but fructose at twice (0.3 kcal/ml) the caloric content of IL. By the end of the 48 h preference test, all the mice switched their preference for fat. Paired t-test, P=1×10⁻⁵, n=7. Note that while at the higher fructose concentration, all of the animals began the test with much stronger attraction to the (sweeter) fructose bottle, all still switched their preference to fat, independent of caloric content.

FIG. 8A, FIG. 8B, FIG. 8C, and FIG. 8D show that strong Fos labelling is observed in neurons of the cNST (Bregma—7.5 mm) in response to ingestion of fat stimuli (FIG. 8C and FIG. 8D), but not in control animals (10% mineral oil, FIG. 8B). Stimulus: 10% linoleic acid (LA), 10% oleic acid (OA). Scale bars, 200 μm. FIG. 8E illustrates the quantification of Fos-positive neurons. ANOVA with Tukey's HSD test against mineral oil (MO, n=5 mice): P=3.4×10⁻⁵ for linoleic acid (LA, n=5 mice), P=3.9×10⁻⁵ for oleic acid (OA, n=5 mice). Values are mean±s.e.m. FIG. 8F, FIG. 8G, FIG. 8H, and FIG. 8I show that TetTox silencing of fat-TRAP cNST neurons does not impair immediate attraction to sweet (3 mM AceK; FIG. 8F and FIG. 8G) or fat (1.5% IL; FIG. 8H, and FIG. 8I). Two-tailed paired t-tests: sweet versus water, wild type, n=10, P=1.1×10⁻⁷; TetTox n=9, P=1.1×10⁻⁶. For fat versus water, wild type, n=10, P=6.8×10⁻⁵; TetTox, n=9, P=1.7×10⁻⁵. Values are mean±s.e.m. j-k, Intragastric infusion with fat activates cNST neurons. FIG. 8J demonstrates that direct intragastric infusion of fat (IL) but not control (PBS) robustly activates the cNST. Scale bars, 100 μm. FIG. 8K shows that quantification of Fos-positive neurons in animals infused with control and IL stimuli, Two-sided Mann-Whitney U-test between control and Intralipid (n=5 mice), P=8×10⁻³. FIG. 8L illustrates that variable labeling in the area postrema is often observed (see FIG. 1C and FIG. 8J), but such labeling is independent of oral versus intragastric infusion. The bar graphs show the quantification of Fos+ neurons in the area postrema (AP) and cNST (FIG. 1D) in response to free licking of IL (90 min) versus intragastric infusion (n=5 mice). cFos in cNST: oral 105±6, infusion 99±6, cFos in AP: oral 93±15, infusion 90±12. The equivalent area of the cNST (bregma—7.5 mm) was processed and counted for the different experiments. Two tailed unpaired t-test, cNST: P=0.54; AP: P=0.86. Values are mean±s.e.m.

FIG. 9A illustrates the methodology for genetic TRAPing of cNST neurons with fat stimuli. Note that animals must not be food deprived to prevent labeling unrelated circuits. The fat-induced TRAP2 neurons were labeled by infection with an AAV carrying a Cre-dependent fluorescent reporter (see corresponding florescence signal), and a second cycle of fat stimulation followed by Fos antibody labelling was performed (see corresponding florescence sign). FIG. 9B allowed the comparison of the number of neurons expressing the fluorescent reporter to the number neurons labelled by Fos antibodies. 90.7±0.6% of Fat-Fos neurons were also TRAPed with the fat stimuli (n=6). Scale bar, 20 μm. FIG. 9C illustrates the targeting of AAV-FLEX-TetTox, or AAV-DIO-mCherry (or GFP) to the cNST for which fat-stimulated TRAP2 animals were used. By comparing the number of neurons expressing AAV after TRAPing and infection, to the number of cNST neurons labeled after crossing similarly TRAped animals to Ai963 reporter mice, the infection of TRAPed neurons is estimated to be >90%: TRAP-AAV: 68±1 neurons; Trap-Ai9: 71±1 neurons (n=8). Scale bar, 100 μm. The equivalent area of the cNST (bregma—7.5 mm) was processed and counted for the separate experiments. Values are mean±s.e.m. FIG. 9D shows a whole mount image of a nodose ganglia from Vip-Cre animals infected with AAV-FLEX-TetTox virus. Average number of labeled neurons from Vip-TetTox was 48±13 neurons (n=4), and the average of nodose neurons labeled with AAV-FLEX-TetTox virus in the Trpa1-Cre animals was 62±23 neurons (n=6; not shown). These numbers compare favorably (˜50%) to the total number of VIP and Trpa1 neurons detected by crossing the Cre animals to reporter Ai963 mice: VIP ˜100 neurons; Trpa1 ˜120 neurons (data not shown). Scale bar, 100 μm. FIG. 9E shows a whole mount image of nodose ganglia from Vip-Cre animals infected with AAV-DIO-hM3Dq (activator DREADD). VIP-DREADD labeling efficiency: 43±4% (43±4/100.5), n=9. Scale bar, 100 μm. FIG. 9F, FIG. 9G, FIG. 9H, and FIG. 9I demonstrates that cNST-activation in response to intestinal delivery of fat and sugar is mediated via vagal signaling. AAV carrying a Cre-dependent GCaMP6s was targeted to the cNST of Penk-Cre animals. FIG. 9F depicts the fiber photometry methodology that was used to monitor cNST activity in response to intestinal delivery of sugar and fat stimuli; to minimize any labeling in the AP and ensure the signals originate in cNST neurons, AAV targeting of GCaMP6s to the cNST was used. FIG. 9G illustrates neural responses following intestinal delivery of fat (10% linoleic acid, LA) or sugar (500 mM glucose, Glu). The light traces denote normalized three-trial averages from individual animals, and the dark trace is the average of all trials. The responses after bilateral vagotomy are shown toward the bottoms of the graph. Black bars below traces indicate the time and duration of stimuli; n=4 mice. NR, normalized response. Note robust, time-locked responses of cNST neurons to intestinal delivery of fat and sugar. Importantly, responses are abolished after bilateral vagotomy. FIG. 9H illustrates the quantification of neural responses before and after vagotomy. Two-tailed paired t-test, P=3.8×10⁻⁵ (sugar), P=5×10⁻⁵ (fat). Data are mean±s.e.m. FIG. 9I shows sample brains of two different injected animals demonstrating expression of GCaMP6s restricted to the cNST, with minimal expression in the AP; the top brain also demarks the location of the recording fiber (dashed rectangle). Scale bars, 200 μm.

FIG. 10A and FIG. 10B Schematic of vagal calcium imaging while simultaneously delivering stimuli into the intestines (see Methods for details). The picture shows a representative view of a vagal nodose ganglion of Vglut-Cre; Ai96 in an imaging session. Two fat responders (denoted #1 and #2) are highlighted, and their responses shown in FIG. 10C, which illustrates sample traces of vagal responses to intestinal stimulation with alternating pulses of vehicle or fat (pre-digested IL). Note time-locked, reliable responses to fat, but not to vehicle control. Stimulus window (60 s) is marked by dotted white lines. Note that since IL is a complex mix, it must be pre-digested in vitro by incubation with lipases prior to using in imaging experiments (versus ingestion, where endogenous lipases in the stomach naturally digest IL). FIG. 10D shows heat maps that depict z-score-normalized fluorescence traces from vagal neurons that responded to pre-digested (dIL, n=79/463 neurons from 8 ganglia). Each row represents the average activity of a single cell to three trials. Stimulus window is shown by dotted white lines. FIG. 10E, FIG. 10F, FIG. 10G, FIG. 10H, and FIG. 10I illustrate heat map responses of vagal neurons to intestinal delivery of a range of fatty acids. FIG. 10E shows z-score-normalized fluorescence traces of vagal neurons to intestinal delivery of 10% LA (10 s) and digested Intralipid (dIL); n=116/634 neurons from 7 ganglia; note that the same neurons responded to both stimuli. FIG. 10F uses 10% LA (10 s) and 10% alpha-linolenic acid (ALA), n=49/322 neurons from 3 ganglia; FIG. 10G uses 10% LA (10 s) and 10% docosahexaenoic acid (DHA), n=51/348 neurons from 5 ganglia; FIG. 10H uses 10% LA (10 s) and 10% oleic acid (OA), n=39/418 neurons from 6 ganglia; FIG. 10I uses 10% LA (10 s) and 10% hexanoic acid (HA), n=52/495 neurons from 6 ganglia.

FIG. 11A is a pie chart illustrating the fraction of fat and sugar responders in the nodose ganglia of Vglut2-GCaMP6s animals. The data is from 1813 neurons from 22 ganglia (red, n=323 cells, 17%). Right, within the responding neurons, 151 (47%) responded to both sugar and fat (“nutrient responders”), while 153 (47%) responded only to fat but not to sugar stimuli (“fat-only responders”). FIG. 11B illustrates sugar/nutrient versus fat-only vagal neurons: Heat maps depicting z-score-normalized vagal responses to intestinal delivery of fat (10% linoleic acid, LA), sugar (500 mM glucose) and amino acids (250 mM amino acid mixture, AA). Each row represents the average activity of a single cell to 3 trials. Stimulus window is shown by dotted white lines. Upper panels show 150 neurons that responded to intestinal application of sugar, fat and amino acids (“sugar/nutrient responders”); bottom panels show 192 neurons that responded only to fat. n=22 ganglia, 1884 imaged neurons. FIG. 11C illustrates representative traces from a “sugar/nutrient responder” (top) and a “fat-only responder” (bottom). Shown are responses to intestinal stimulation with 9 interleaved pulses of fat (10% LA, 10 s, green dotted line), sugar (500 mM Glu, 10 s) and amino acids (250 mM AA, 60 s). FIG. 11D shows heat maps of the small subset of vagal neurons that responded to sugar and amino acids but not to fat (n=14/1884 neurons from 22 nodose ganglia). On average, less than 1 neuron was detected per ganglia. When using high concentrations of glucose stimuli (>250 mM) for long stimulation times (60 s) one can detect strong vagal responses, but these have been shown not to be sugar-preference responses.

FIG. 12A depicts the methodology of vagal calcium imaging while simultaneously delivering sugar and fat stimuli into the intestines. The bottom inset illustrates CCK-expressing enteroendocrine cells (EECs) in the intestines. For FIG. 12B, the role of glutamate signaling was examined by imaging the responses of the relevant sugar preference vagal neurons to intestinal sugar stimuli before and after addition of a mixture of AP3 and KA glutamate receptor antagonists. Indeed, the results demonstrated that pharmacological inhibition of glutamate-based signaling has no effect on this gut-to-vagal sugar/nutrient sensing circuit. Shown are representative traces of vagal neuron responses to intestinal infusions of fat, sugar and amino acids before and after treatment with ionotropic/metabotropic glutamate receptor antagonists (2 mg/kg AP3 with 300 μg/kg kynurenic acid, see Methods). Top traces show sugar/fat/amino acid responding vagal neurons, bottom traces show fat-only responders. FIG. 12C, in contrast, shows that pharmacological inhibition of glutamate-based signaling abolished all osmolarity responses. Heat maps depicting z-score-normalized vagal responses to intestinal osmolarity stimuli (60 s of 1 M NaCl) before and after treatment with ionotropic/metabotropic glutamate receptor antagonists (2 mg/kg AP3 with 300 μg/kg kynurenic acid). FIG. 12D shows quantification of the responses to 1 M NaCl, 10% LA. 500 mM Glucose, and 250 mM AA mixture before (left bars in each group) and after blockers (right bars in each group). 1 M NaCl, n=56 neurons, P=1×10⁻¹⁰. For nutrient responders: LA, n=21, P=0.16; Glucose, n=21, P=0.85; AA, n=21, P=0.07. For fat-only responders, n=19, P=0.54 by two-tailed paired t-tests. All values are mean±s.e.m. AUC: average area under curve. FIG. 12E (left) shows representative traces of vagal neuron responses to intestinal infusions of fat, sugar and amino acids before and after treatment with holecystokinin A receptor (CCKAR) blocker (4 mg/kg devazepide). Note robust, reliable responses to fat (10% LA) and sugar (500 mM Glucose) prior to addition of CCKAR antagonist. However, all responses are loss after addition of antagonist (top panel). By contrast, fat-only responses are unaffected (bottom panel). The right bar graph shows the quantification of responses before (open bars) and after (right bar in each group) CCKAR antagonist (data from FIG. 3A and FIG. 3B). For nutrient responders: LA, n=37 neurons, P=1×10⁻⁹; Glucose, n=37, P=1×10⁻⁹; AA, n=37, P=1×10⁻⁹. For fat only responders, n=38, P=0.11 by two-tailed paired t-tests. All values are mean±s.e.m. FIG. 12F illustrates sugar/nutrient but not fat-only responders utilize CCK signaling. Left, Heat maps depicting z-score-normalized fluorescence traces from vagal neurons identified as sugar/nutrient responders (upper panels, n=41 neurons); note responses to sugar, fat and amino acid stimuli. The lower heat-map shows the fat-only neurons (n=41 neurons). After stimulating with CCK (1 μg/ml), all sugar/nutrient responders were activated, but not the fat-only vagal neurons. Right, Representative traces of 2 sample neurons to pulses of 10% linoleic acid (LA), 500 mM glucose (G), 250 mM amino acids (A), and CCK. Stimulus windows are indicated by dotted lines. FIG. 12G and FIG. 12H show CCK-dependent (sugar and fat) and CCK independent (fat-only) intestinal gut-to-brain fat-preference pathways co-contribute to fat signals in the cNST. Shown are photometric recordings of cNST neurons in Penk-Cre animals in response to intestinal fat-evoked activation of both fat-stimulated vagal pathways (black traces and bars). Shown in right graphs of FIG. 12H are the same responses after inhibiting signaling via the CCK-dependent vagal pathway. FIG. 12I demonstrates that cNST responses to intestinal fat stimulation are reduced to ˜50% after removing CCK-dependent signaling, highlighting the separate contributions of the two fat-preference circuits. As expected, sugar-evoked responses are completely abolished after inhibiting signaling via the CCK dependent pathway. n=5, P=2.4×10⁻⁶ by two-tailed paired t-test. All values are mean±s.e.m. In gain-of-function experiments, with DREADD being overexpressed in vagal neurons, activation of a single pathway is sufficient to create new preferences.

FIG. 13A shows a tSNE plot of the transcriptome of mouse vagal nodose neurons (original data set taken from: Bai, L., et al., Genetic identification of vagal sensory neurons that control feeding. Cell 179, 1129-1143.e1123 (2019).); CCKAR expression is represented on the scale. FIG. 13B demonstrates that CCKAR-expressing neurons respond to intestinal stimulation with nutrients. An engineered Cckar-iCre was used to drive GCaMP6s expression in CCKAR vagal neurons. 724 imaged neurons from 12 ganglia were analyzed. Shown are heat maps depicting z-score normalized fluorescence traces of the CCKAR-expressing neurons responding to intestinal delivery of fat (10% linoleic acid), sugar (500 mM glucose) or amino acids (250 mM amino acid mixture). Stimulus window is shown by dotted white lines. FIG. 13C shows a tSNE plot of the transcriptome of mouse vagal nodose neurons; urotensin 2B (Uts2b) expression is represented on the scale. FIG. 13D shows responses of vagal Uts2b-expressing neurons (Uts2b-GCaMP6s) to intestinal delivery of fat (10% linoleic acid), sugar (500 mM glucose) or amino acids (250 mM amino acid mixture). The heat maps depict z-score-normalized fluorescence traces of sugar/nutrient responders (n=52/207 neurons from 7 ganglia). Stimulus window is shown by dotted white lines. Note that only 3 of the 52 neurons responded only to fat (shown at the top of the heat maps). FIG. 13E demonstrates that sugar/nutrient responders are a unique subset of CCKAR-expressing vagal neurons. Heat maps showing z-score-normalized fluorescence traces from vagal neurons that respond to CCK and nutrient stimuli. While all of the neurons that responded to intestinal stimulation with sugar, fat and amino acids (i.e., the sugar/nutrient sensors) also responded to CCK, the vast majority of vagal neurons that respond to CCK do not respond to nutrient stimuli (bottom heat maps, n=136 neurons). This is expected since only a small fraction would be mediating sugar/nutrient preference, versus other roles of CCK signaling. Stimuli: 10% linoleic acid (LA), 10 s; 500 mM glucose, 10 s; 250 mM amino acids (AA), 60 s; 1 μg/ml CCK, 60 s. FIG. 13F is a pie chart based on data from 12 ganglia. Since vagal neurons that only respond to fat stimuli are not activated by CCK, they are not part of this analysis. FIG. 13G shows pie charts depicting the fraction of sugar/nutrient and fat-only responders in animals driving GCaMP6s reporter from various driver lines: Vglut2-Cre, Cckar-Cre, Vip-Cre, Uts2b-Cre, and Trpa1-Cre animals. VIP/Uts2b define the sugar/nutrient responders while TrpA1 mark the fat-only responders.

FIG. 14A shows graphs for consumption (AceK and IL) in two-bottle 48 h preference assay for control and cNST-silenced animals (n≥8 mice), P=0.151 (from FIG. 2A). FIG. 14B illustrates consumption in two-bottle 48 h preference assay for control and Vip-silenced mice (n≥7 mice), P=0.69. FIG. 14C illustrates consumption in two-bottle 48 h preference assay for control and Trpa1-silenced mice (n≥6 mice), P=0.44. Values are mean±s.e.m. FIG. 14D, FIG. 14E, and FIG. 14F demonstrate that animals with genetically silenced sugar/nutrient preference vagal neurons (VIP), or fat-only vagal neurons (Trpa1) still exhibit normal innate attraction to sweetener (FIG. 14D), sugar (FIG. 14E), and fat stimuli (FIG. 14F). Shown are graphs for 30 min two-bottle tests for control mice, and for mice with silenced VIP-expressing vagal neurons (VIP-Tx) and mice with silenced Trpa1-expressing vagal neurons (Trpa1-Tx). FIG. 14D shows that AceK versus water in VIP-Tx (n=8) and Trpa1-Tx (n=6) animals are not significantly different from controls. ANOVA with Tukey's test: VIP-Tx, P=0.36, Trpa1-Tx, P=0.66. FIG. 14E shows glucose versus water in VIP-Tx (n=8) and TrpA1-Tx (n=6) is not significantly different from control animals. ANOVA with Tukey's test: VIP-Tx, P=0.45, Trpa1-Tx, P=0.67. FIG. 14F shows IL versus water in VIP-Tx (n=8) and TrpA1-Tx (n=6) is not significantly different from control animals. ANOVA with Tukey's test: VIP-Tx, P=0.87, Trpa1-Tx, P=0.91. Values are mean±s.e.m. Tastants: AceK (3 mM), Glucose (200 mM), IL (1.5%). FIG. 14G is a graph that shows body weight measurements from VIP-Cre animals injected with AAV-Flex-TetTox in both nodose ganglia, from the time the animals were infected until the time behavioral preference tests were performed (days 24-26.); data is presented as percent change, with weight at time zero defined as 100%. Thin lines represent individual animals; dark lines represent the average body weight of TetTox (n=7 mice, closed circles) and control (n=10 mice, open circles) animals. No significant differences were detected, two-way ANOVA, P=0.37.

FIG. 15A shows double in situ hybridization labeling for the endogenous Trpa1 gene (left) and for Cre-recombinase (middle) in the nodose of Trpa1-Cre knock-in mice. Shown is a frozen section demonstrating the strong overlap (n=3 mice). The left 3 panels show the in situ results, and the right 3 panels show an illustration of the labeling results. Scale bars, 100 μm. FIG. 15B, FIG. 15C, FIG. 15D, and FIG. 15E show tSNE plots of the nodose transcriptome highlighting the 4 clusters, and heat maps of responses to intestinal delivery of fat and sugar from various vagal clusters using the corresponding Cre driver lines. Gpr65, Piezo2, Calca, and Oxtr vagal neurons do not sense fat or sugar. FIG. 15B shows that GPR65 vagal neurons are known to indiscriminately respond to a wide range of long stimuli at high concentrations, including salt, fructose, mannose, and glucose, and considered osmolarity responders. The heat maps show z-score-normalized fluorescence traces from all imaged vagal neurons in response to intestinal infusions of fat (10% linoleic acid, LA, 10 s), sugar (500 mM glucose, 10 s) or high osmolarity salt (1 M NaCl) for 60 s in Gpr65-Cre; Ai96 animals. Each row represents the average activity of a single cell to three trials. Stimulus window is shown by dotted white lines. n=69 neurons from 3 ganglia. FIG. 15C, FIG. 15D, and FIG. 15E depict calcium imaging of vagal responses in Piezo2-Cre; Ai96, Calca-Cre; Ai96, and Oxtr-Cre; Ai96 animals. The heat maps showing z-score-normalized fluorescence traces of all imaged neurons in response to intestinal infusion of fat or sugar. Piezo2: n=99 neurons from 4 ganglia (FIG. 15C); Calca: n=168 neurons from 5 ganglia (FIG. 15D); Oxtr: n=89 neurons from 6 ganglia (FIG. 15E). No significant responses were detected for any of the lines. FIG. 15F illustrates the generation of fat receptor knockouts. Schematic illustrating the structural domains of the murine wild type CD36, GPR40, and GPR120 protein sequences, with the deletions denoted by the black boxes. For CD36 KO, a 626 nucleotide (nt) deletion was engineered removing residues 107 to 185, which forms part of the hydrophobic binding pocket of CD3644. For GPR40 KO, a 695 nt deletion was engineered that removed more than 75% of the protein. For GPR120, a 412 nt deletion was introduced removing 136 residues, and introducing a nonsense frameshift disrupting functional translation of the remaining two-thirds of the protein. FIG. 15G shows representative views of GCaMP6s expressing neurons in vagal nodose imaging sessions using Vglut2-Cre; Ai96 animals (left) or Snap25-GCaMP6s animals (right). Scale bar, 100 μm. Similar results were obtained from multiple animals. FIG. 15H compares the fraction of sugar/nutrient responders (left) or fat-only responders (right), between Vglut2-Cre; Ai96 (Vglut2-G6s, black, n=22 ganglia) and Snap25-GCaMP6s animals (Snap25-G6s, red, n=7 ganglia). No significant differences were found in vagal responses to intestinal delivery of fat or sugar between the Vglut2-G6s and Snap25-Gos genetic drivers (Two-sided Mann-Whitney U-test, P=0.29 for sugar/nutrient responders, P=0.83 for fat-only responders). All values are mean±s.e.m.

FIG. 16A, FIG. 16B, FIG. 16C, FIG. 16D, FIG. 16E, and FIG. 16F illustrate the functional imaging of vagal neurons in response to intestinal delivery of fat (10% linoleic acid) and sugar (500 mM glucose) in Snap25-GCaMP6s mice harboring various combinations of fat receptor deletions. Heat maps show sugar/nutrient responders (top panels), and fat-only responders (bottom panels). Control (n=7 ganglia) (FIG. 16A); CD36 KO (n=6 ganglia) (FIG. 16B); GPR40 KO (n=7 ganglia) (FIG. 16C); GPR120 KO (n=6 ganglia) (FIG. 16D); CD36 & GPR40 double KO (n=6 ganglia) (FIG. 16E); CD36 & GPR120 double KO (n=8 ganglia) (FIG. 16F). FIG. 16G compares vagal responses to intestinal sugar stimuli in all fat receptor knockouts. ANOVA with Tukey's HSD test to WT (n=7): CD36 KO (n=6 mice), P=0.99; GPR40 KO (R40, n=7 mice), P=0.87; GPR120 KO (R120, n=6 mice), P=0.94; CD36/GPR40 double KO (CD36/R40, n=6 mice), P=0.99; CD36/GPR120 double KO, (CD36/R120, n=8 mice), P=0.96; GPR40/GPR120 double KO (R40/120, n=7 mice), P=0.99; CD36/GPR40/GPR120 triple KO (3KO, n=6 mice), P=0.99. Values are mean±s.e.m. FIG. 16H demonstrates that fat receptor knockout animals that cannot transmit the gut-brain signal (GPR40/GPR120 double knockouts, and the triple knockout) still exhibit normal innate attraction to fat stimuli. Shown are brief-access (30 min) two-bottle tests for artificial sweetener (3 mM AceK) versus water (left panel), and fat (1.5% Intralipid, IL) versus water (right panel). ANOVA with Tukey's test compared to wild type sweet consumption (n=9): GPR40/GPR120 double KO (R40/R120): n=6, P=0.96; CD36/GPR40/GPR120 triple KO (D36/R40/R120): n=5, P=0.26. ANOVA with Tukey's test compared to wild type fat consumption, R40/R120: n=6, P=0.25; n=5, D36/R40/R120: P=0.98. Two-tailed paired t-test. Values are mean±s.e.m.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub combination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

The term “subject” as used herein is intended to mean any animal, in particular, mammals. Although methods of regulating preference for fat and/or sugar is exemplified in mice herein, the nutrient preferences of any type of mammal, including human and nonhuman mammals, can be regulated using the disclosed methods. Further, although methods of screening a substance for an ability to activate a nutrient receptor in the gut is exemplified in mice herein, the screening of substances can be assessed in any type of mammal using the disclosed methods. “Subject” and “patient” are used interchangeably herein.

Disclosed herein are methods of regulating a subject's preference for fat and/or sugar comprising administering to the subject a molecule that activates or inhibits one or more of a GPR40 receptor, a GPR120 receptor, and a CCK signaling pathway.

The disclosed methods can be used to decrease the subject's preference for fat and/or sugar. In some embodiments, the disclosed methods decrease the subject's preference for fat. In some embodiments, the disclosed methods decrease the subject's preference for sugar. In some embodiments, the disclosed methods decrease the subject's preference for fat and sugar.

The disclosed methods can be used to increase the subject's preference for fat and/or sugar. In some embodiments, the disclosed methods increase the subject's preference for fat. In some embodiments, the disclosed methods increase the subject's preference for sugar. In some embodiments, the disclosed methods increase the subject's preference for fat and sugar.

Suitable receptors upon which the molecule can act include GPR40 and GPR120. In some embodiments, the molecule activates or inhibits GPR40. In some embodiments, the molecule activates or inhibits GPR120. In other embodiments, the molecule activates or inhibits GPR40 and GPR120.

The molecule can activate the GPR40 receptor and/or the GPR120 receptor. The molecule can inhibit the GPR40 receptor and/or the GPR120 receptor. The molecule can activate or inhibit GPR40 and/or GPR120 directly or the downstream signaling pathway of GPR40 and/or GPR120. In some embodiments, the molecule can activate or inhibit the downstream signaling pathway of GPR40 and/or GPR120. In some embodiments, the molecule activates or inhibits the downstream signaling of GPR40. In some embodiments, the molecule activates or inhibits the downstream signaling of GPR120. In other embodiments the molecules activates or inhibits the downstream signaling of GPR40 and GPR120.

The molecule that activates GPR40 and/or GPR 120 or the downstream signaling pathway of GPR40 and/or GPR120 can increase the subject's fat preference. The molecule that inhibits GPR40 and/or GPR120 or the downstream signaling pathway of GPR40 and/or GPR 120 can decrease the subject's fat preference. The increase or decrease in fat preference can comprise activation or inhibition of the combination of receptors.

The molecule can activate CCK signaling from gut nutrient receptors to the brain. Suitable activators of CCK signaling include peptides, such as CCK peptide, and a small molecules.

The molecule can inhibit CCK signaling from gut nutrient receptors to the brain. Suitable inhibitors of CCK signaling include Devazepide, a small molecule, or a peptide.

The disclosed methods can result in increased activation of vagal sensory neurons following activation of gut nutrient receptors. Alternatively, the methods can result in decreased activation of vagal sensory neurons following inhibition of gut nutrient receptors. Vagal sensory neurons capable of increased or decreased activation in response to gut receptor signals include vagal sensory neurons that express TRP1, CCKAR, Uts2b, or vasoactive intestinal peptide (VIP).

Also provided herein are methods of screening a substance for an ability to activate or reduce activation of a nutrient receptor in a subject's gut. The methods comprise administering a substance to a subject wherein the subject's vagal sensory neurons express a genetically encoded calcium indicator (GECI); imaging the vagal sensory neurons of the subject; and detecting a presence or absence of a signal from the GECI, wherein the presence of the signal in a region of the subject's vagal sensory neurons that are responsive to nutrient receptor activation in the gut indicates that the substance activates the nutrient receptor. Alternatively, a reduction of the signal in a region of the subject's vagal sensory neurons that are otherwise responsive to nutrient receptor activation in the gut indicates that the substance inhibits the nutrient receptor.

Suitable routes of administering the substance include, for example, oral and intestinal infusion. In some embodiments the substance is administered orally. In other embodiments the substance is administered by intestinal infusion.

Following administration of a substance, activation of vagal sensory neurons of the subject can be detected via imaging and the presence or absence of a signal from the GECI is assessed. Activation of the subject's vagal sensory neurons that are responsive to nutrient receptor activation in the gut indicates that the substance activates the nutrient receptor. In some embodiments, the presence of the signal in the vagal sensory neurons indicates that a substance has activated a nutrient receptor in the gut.

The absence of signal in the vagal sensory neurons can indicate that a substance has not activated a nutrient receptor in the gut under conditions where the vagal neurons that are responsive to nutrient receptor activation are not otherwise activated. In conditions where vagal neurons that are responsive to nutrient receptor activation are activated in the absence of the substance, a reduction in vagal neuron activation following administration of the substance can indicate that the substance reduces activation of or inhibits a nutrient receptor in a subject's gut.

Suitable neurons to be tested for activation following activation of a gut nutrient receptor by a substance include neurons that express TRPA1, CCKAR, Uts2b, or vasoactive intestinal peptide (VIP).

Suitable caloric substances can comprise fats, sugars, amino acids, or a combination thereof. Other suitable substances include noncaloric or low caloric compounds. In some embodiments, the substance can comprise both caloric compounds and non- or low-caloric compounds.

Suitable receptors to be tested for activation or inhibition following administration of a substance include GPR40 and/or GPR120. In some embodiments, the substance activates or inhibits GPR40. In some embodiments, the substance activates or inhibits GPR120. In other embodiments, multiple receptors are activated or inhibited by the substance. In some embodiments, the substance activates or inhibits GPR40 and GPR120. The methods can comprise administration of a substance that activates or inhibits any combination of receptors. In other embodiments, the substance activates or inhibits CCK signaling pathway. In other embodiments, the substance activates or inhibits TRPA1 vagal neuron pathway. In other embodiments the substance activates or inhibits CCK signaling pathway and TRPA1 vagal neuron pathway.

### EXAMPLES

### Results

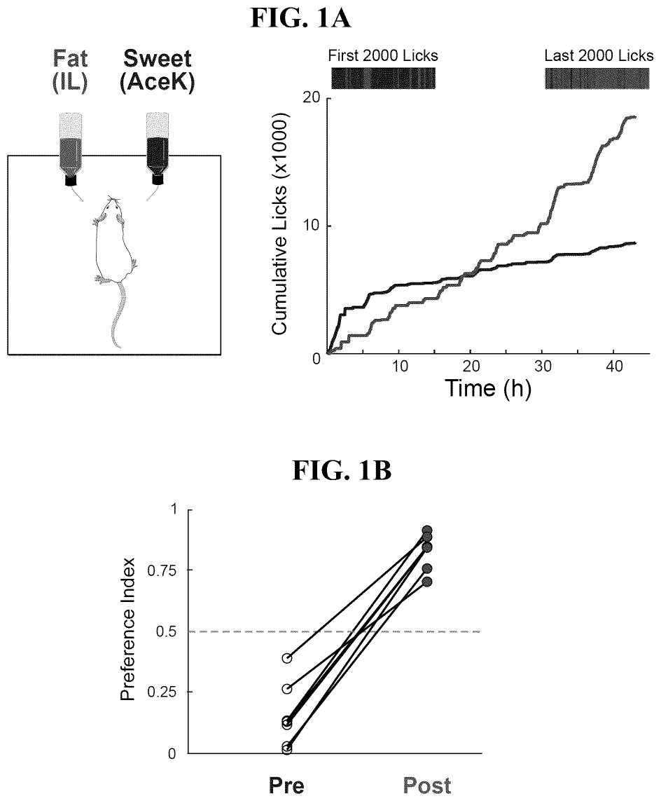

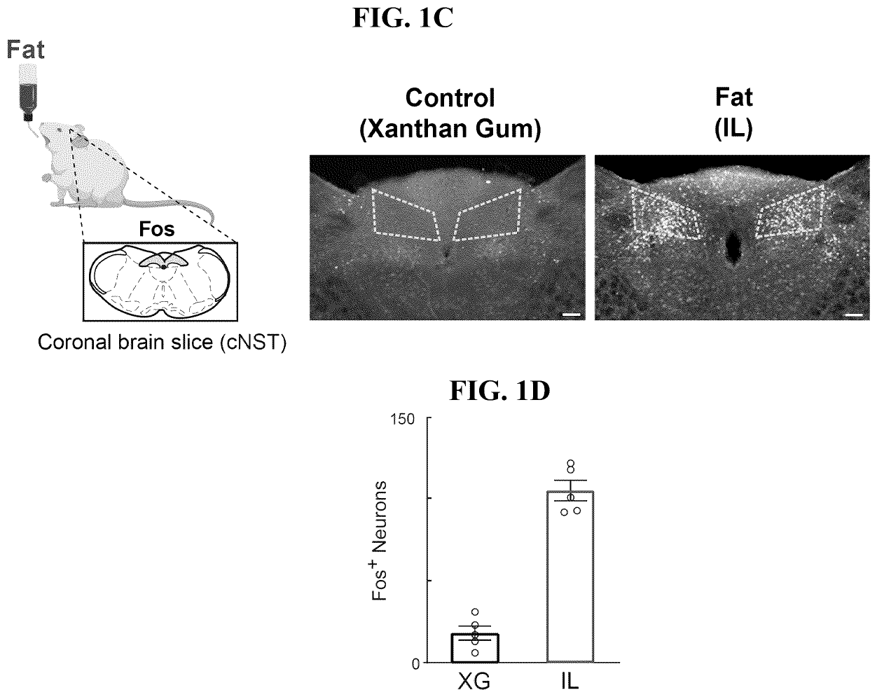

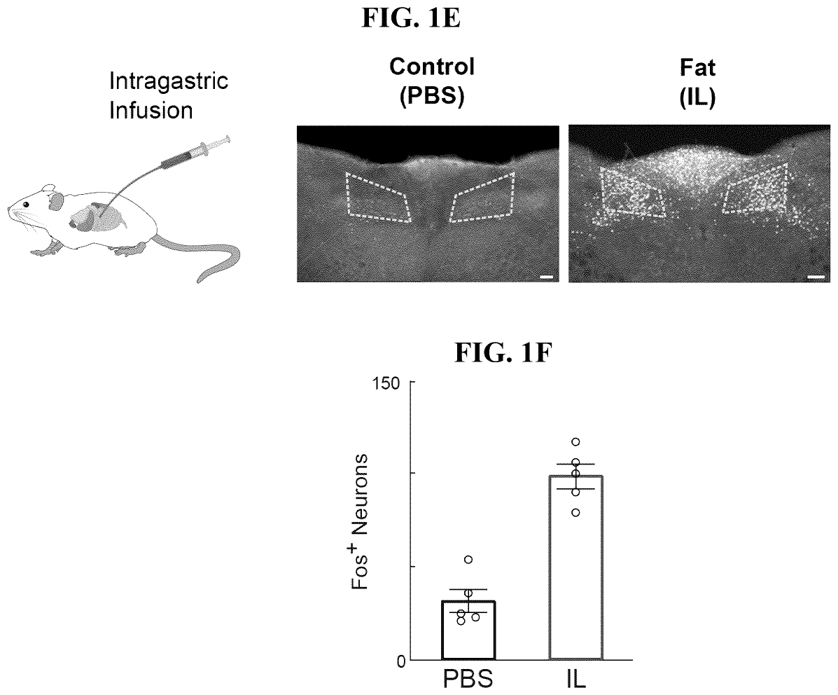

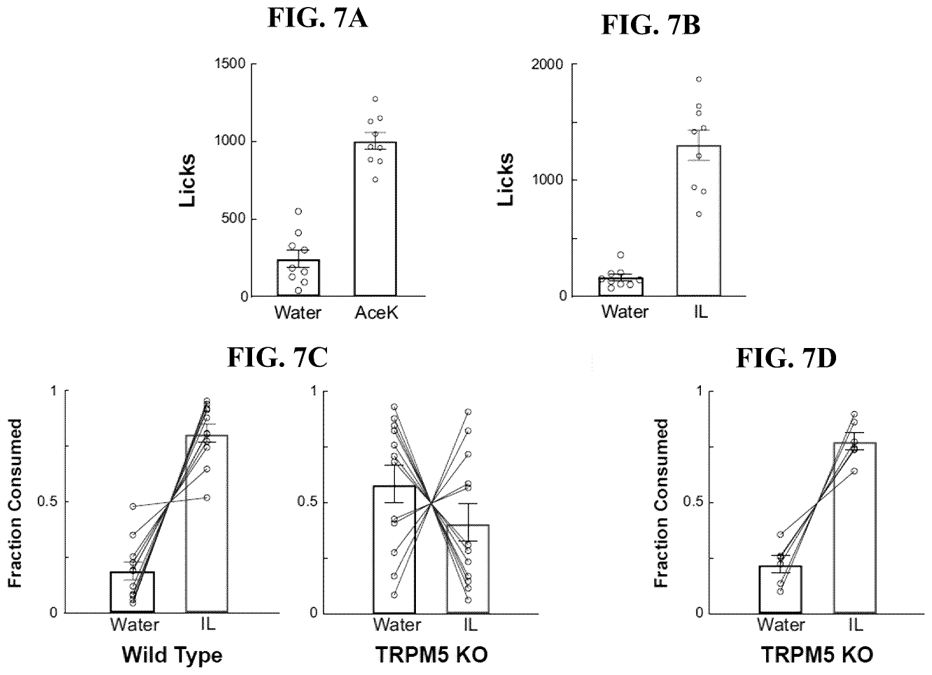

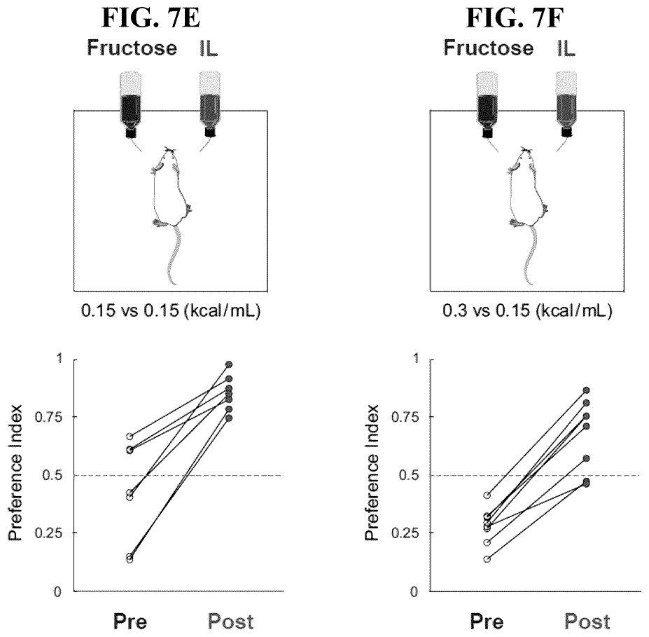

The Development of Fat preference. To behaviorally monitor the development of fat preference, animals were presented with a choice between an artificial sweetener (3 mM acesulfame K, AceK) and fat (1.5% Intralipid, IL) (FIG. 1A). Both stimuli are innately attractive to a naive animal (FIG. 7A, FIG. 7B). Because artificial sweeteners do not trigger post-oral preference, however, the analysis of the emergence of fat preference without the confound of having two preference-creating stimuli was possible. Indeed, although animals initially preferred the artificial sweetener (FIG. 1A, FIG. 1B), within 24 h of exposure to both choices, their preference is markedly altered, such that by 48 h, the mice drank almost exclusively from the bottle containing fat (FIG. 1A, FIG. 1B). This behavioral switch illustrates the ability of fat stimuli to induce strong consummatory responses and appetitive behavior.

It has been suggested that the immediate attraction to fat (rather than the long-term preference) is dependent on the TRPM5 channel expressed in taste receptor cells (FIG. 7C). If the development of fat preference is mediated via post-ingestive signaling, it should be independent of TRPM5 function, and consequently TRPM5 knockout animals should still be capable of developing behavioral preference for fat. Indeed, TRPM5 mutant animals, while blind to the taste of fat, still are fully capable of developing strong post-ingestive preference for fat (FIG. 7D).

Fat preference is mediated via the gut-brain axis. For an animal to develop a preference for fat over sweetener, it must distinguish between two innately attractive stimuli. Identification of a population of brain neurons that respond selectively to the consumption of fat would provide an entry to reveal the neural control of fat preference and the basis for the insatiable appetite for fat.

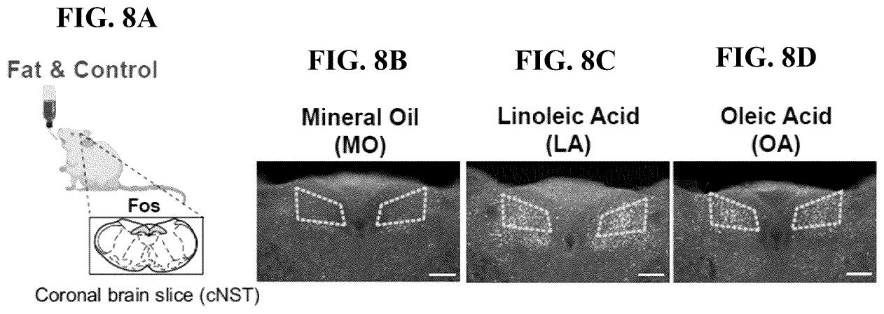

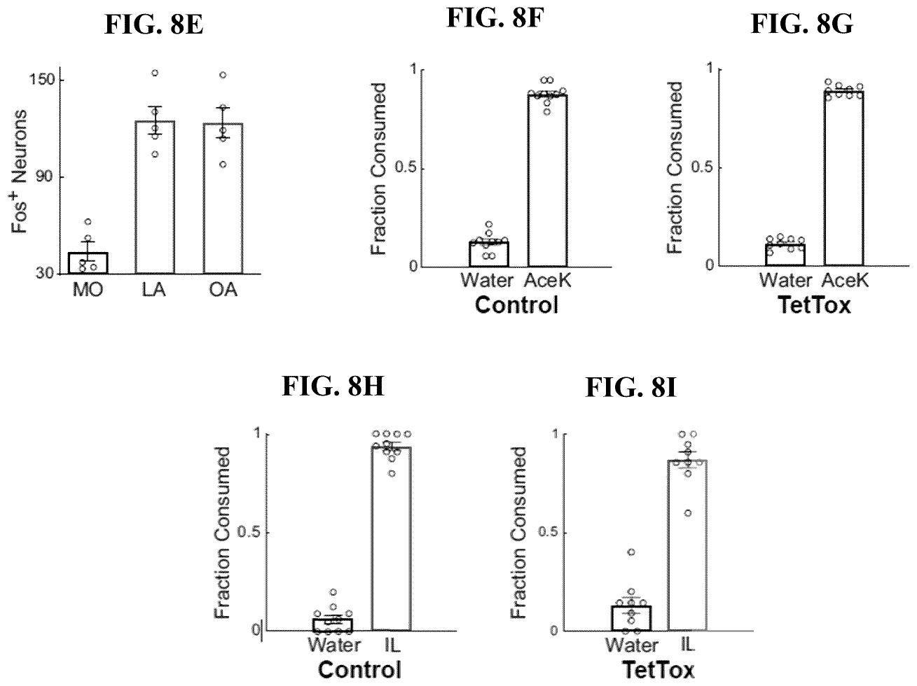

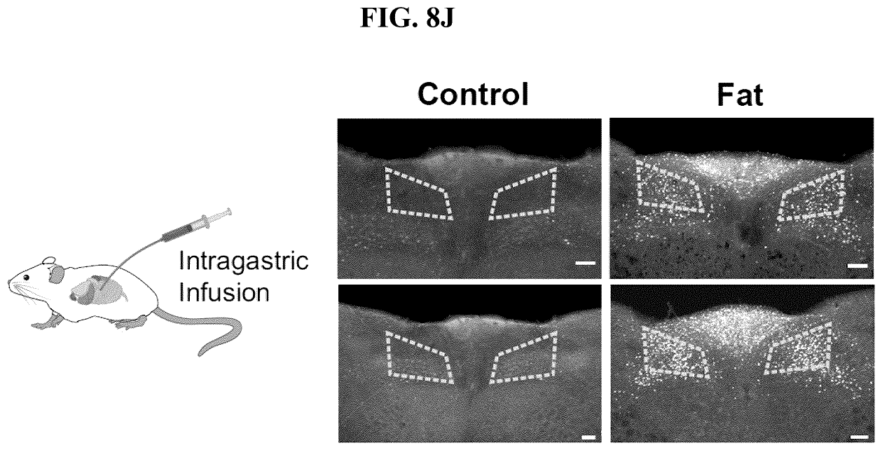

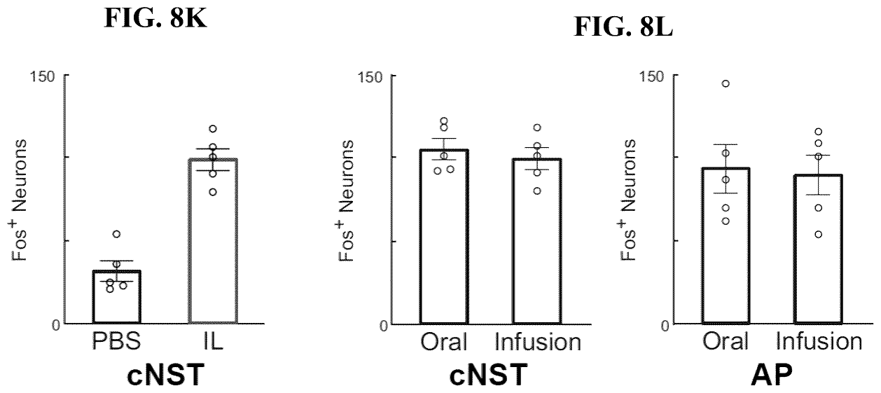

Separate cohorts of animals were exposed to 3 different lipid stimuli (Intralipid, linoleic acid, or oleic acid) and to fat-free textural controls (xanthan gum or mineral oil). Using Fos as a proxy for neural activity, fat, but not control stimuli, elicited strong bilateral activation of neurons in the caudal Nucleus of the Solitary Tract (cNST) in the brainstem (FIG. 1C, FIG. 1D, FIG. 8A, FIG. 8B, FIG. 8C, FIG. 8D, FIG. 8E). The cNST is a nexus of interoceptive signals conveying information from the body to the brain via the gut-brain axis. If the fat-activated brain cNST neurons are receiving signals originating in the gut, then direct delivery of fat stimuli into the gut should also induce activation of the cNST. An intragastric catheter was implanted in the stomach and infused either a fat solution or a vehicle control. Intragastric infusion of fat, but not of a vehicle, is sufficient to activate the cNST as robustly as oral ingestion (FIG. 1E, FIG. 1F, FIG. 8J, FIG. 8K, FIG. 8L).

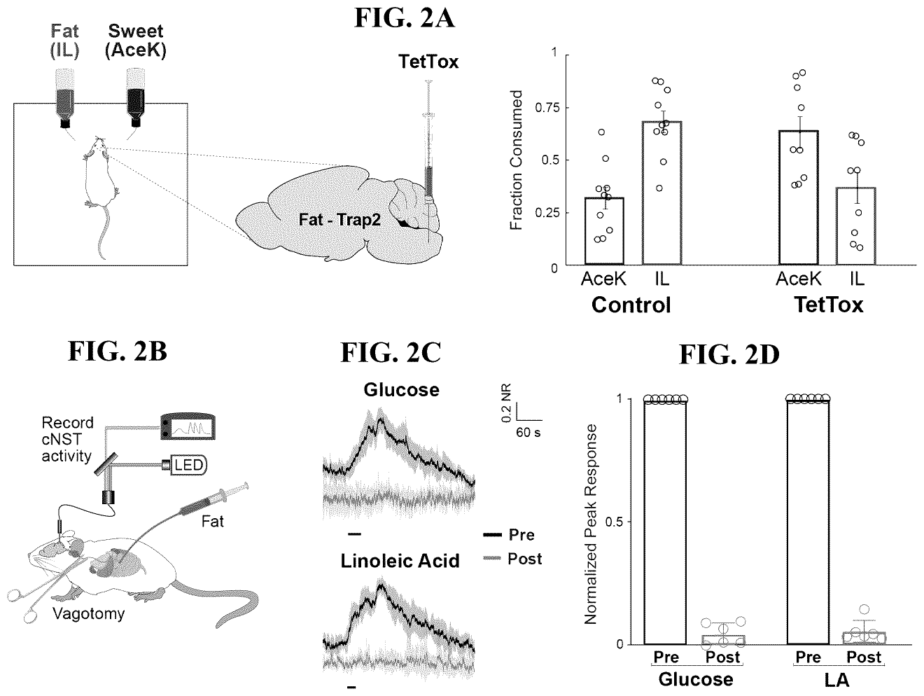

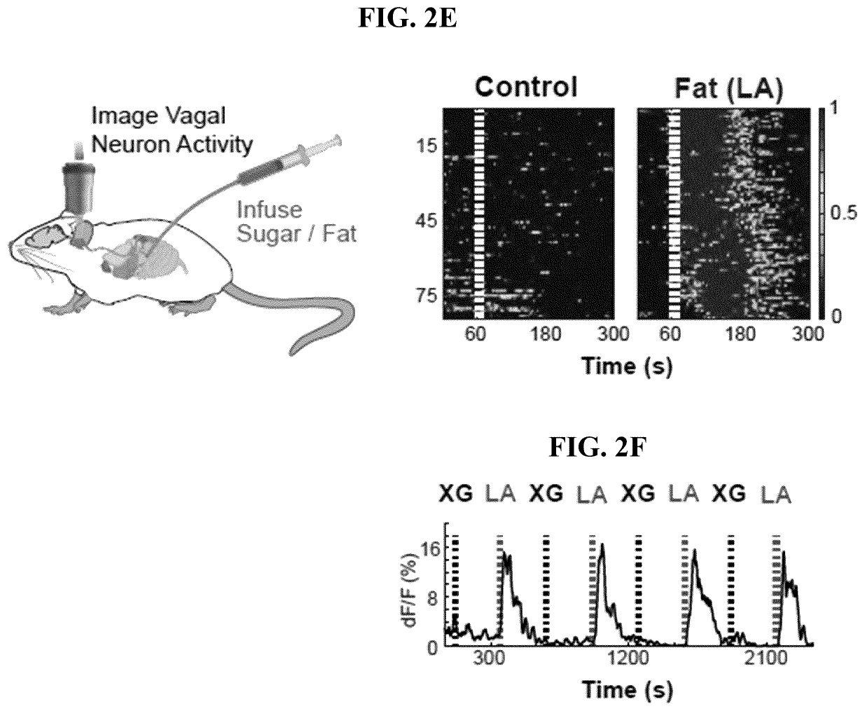

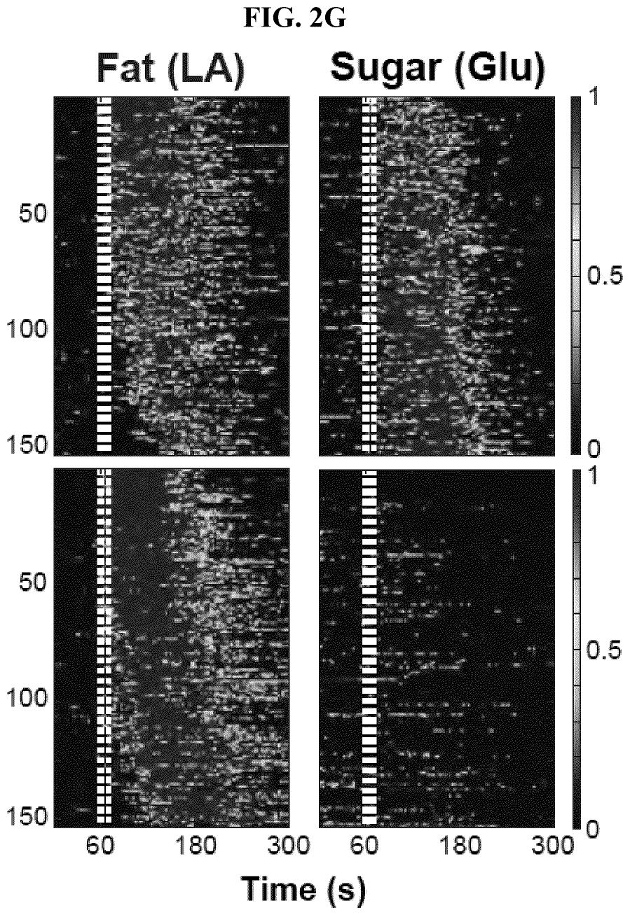

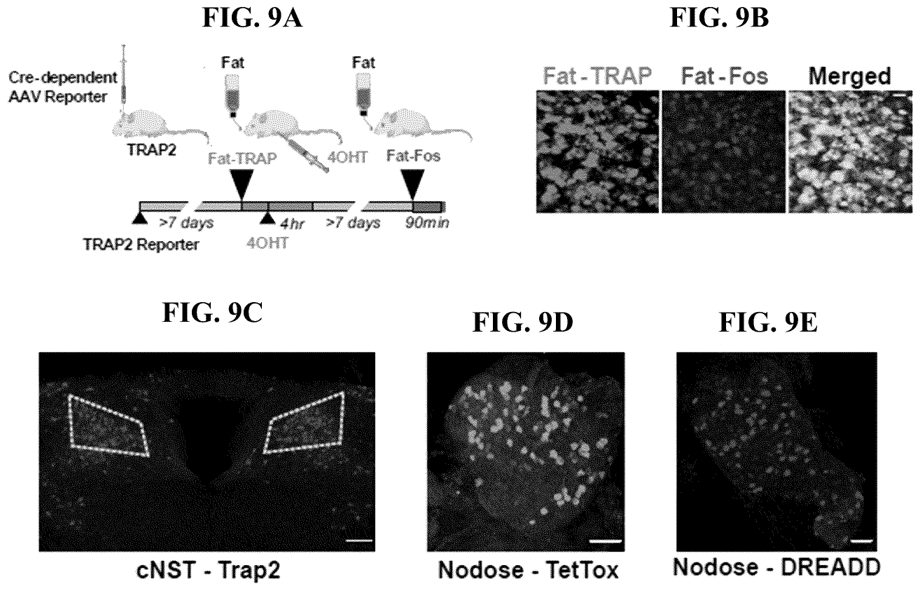

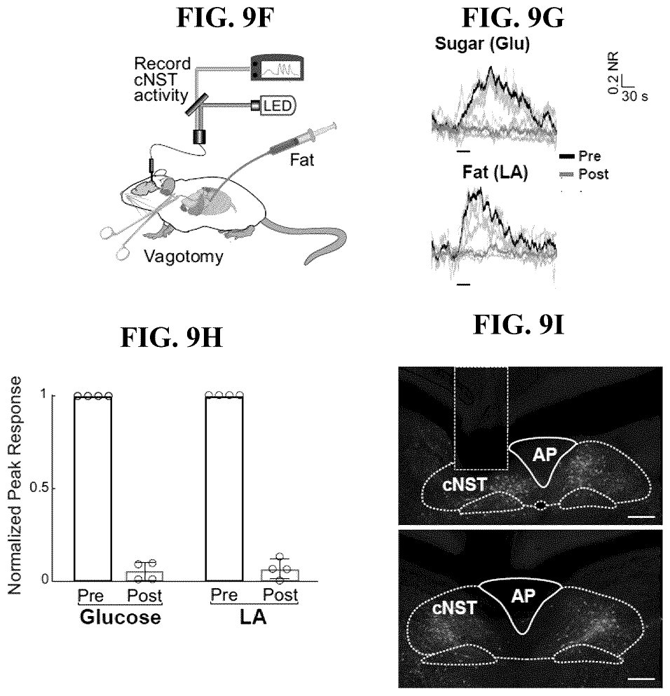

If the fat-activated cNST neurons are essential for creating fat preference, then blocking their function should prevent the development of fat preference. Targeted recombination in active populations (TRAP) system was used to target Cre recombinase to fat-activated cNST neurons (fat-TRAPed), and then bilaterally injected an adeno-associated virus (AAV) carrying a Cre-dependent tetanus toxin light chain (TetTox) construct, so as to genetically silence synaptic transmission in the cNST neurons responding to fat (FIG. 2A, FIG. 9A, FIG. 9B, FIG. 9C, FIG. 9D, FIG. 9E, FIG. 9F, FIG. 9G, FIG. 9H, FIG. 9I).

To ensure that the genetic silencing did not affect the animals' taste-dependent, immediate attraction to fat, they were first tested in a standard fat versus water discrimination assay. Indeed, they drank preferentially from the fat solution, and were indistinguishable from controls (FIG. 8F, FIG. 8G, FIG. 8H, FIG. 8I). By contrast, silencing the fat-activated cNST neurons abolished the development of post-ingestive preference for fat, even after prolonged testing sessions (FIG. 2A).

Fat and sugar activate vagal neurons. To investigate how fat signals are transferred from the gut to the brain, fat stimuli were infused into the gut, and used fibre photometry to simultaneously record neural activity in cNST neurons (FIG. 2B). cNST neurons are robustly activated by direct intestinal infusion of fat, with responses closely tracking the delivery of the stimulus (FIG. 2C, FIG. 2D, FIG. 9F, FIG. 9G, FIG. 9H, FIG. 9I). The cNST also responded to direct infusion of sugar (FIG. 2C, FIG. 2D).

The vagus nerve serves as a key conduit for conveying information from the gut to the brain. If the vagus nerve is required for the transmission of fat signals from the gut to the cNST, then transection of the vagus nerve should prevent the signals from reaching the brain. To test this hypothesis, the gut was infused with fat (or sugar as a control) and the stimulus-evoked responses were recorded. Indeed, fat-activated neural responses in the cNST were effectively abolished after bilateral vagotomy (FIG. 2C, FIG. 2D, FIG. 9F, FIG. 9G, FIG. 9H, FIG. 9I), thus establishing the vagus nerve as the conduit for transmitting the fat signal from the gut to the brain.

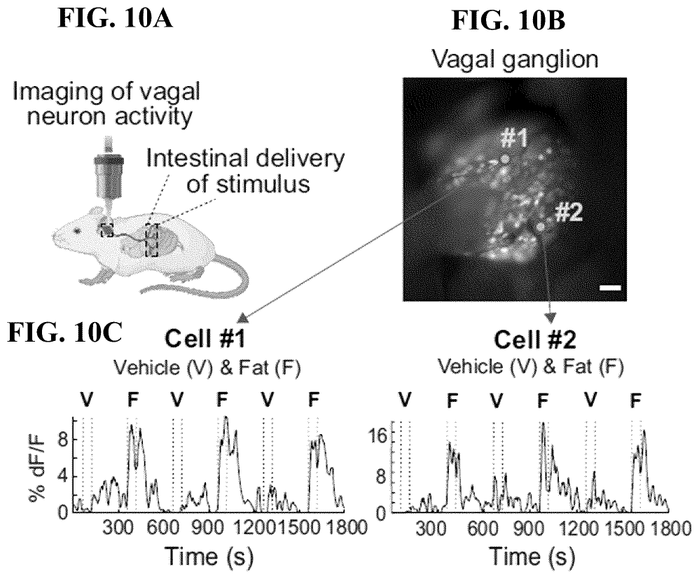

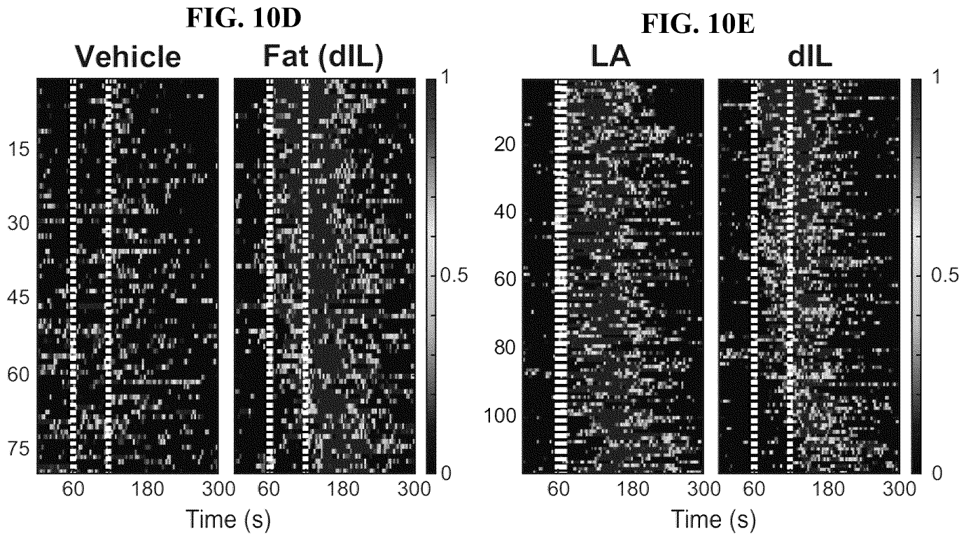

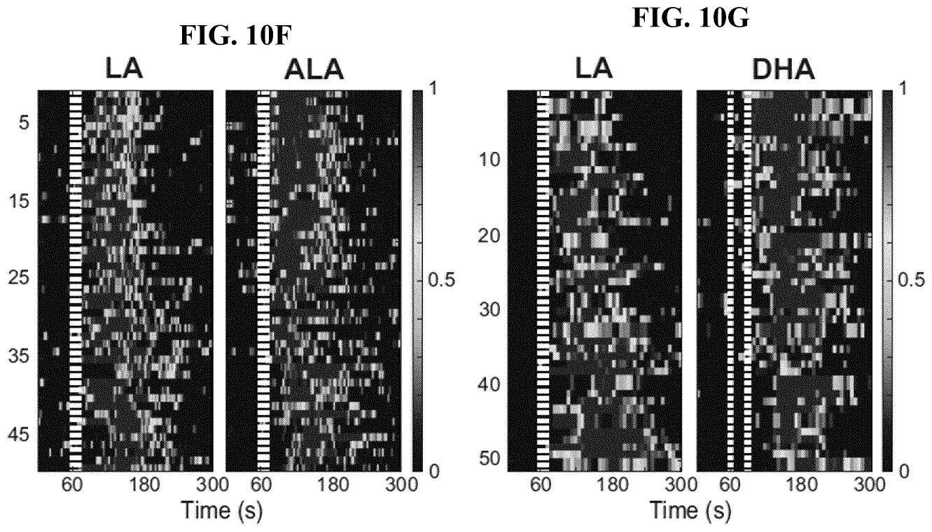

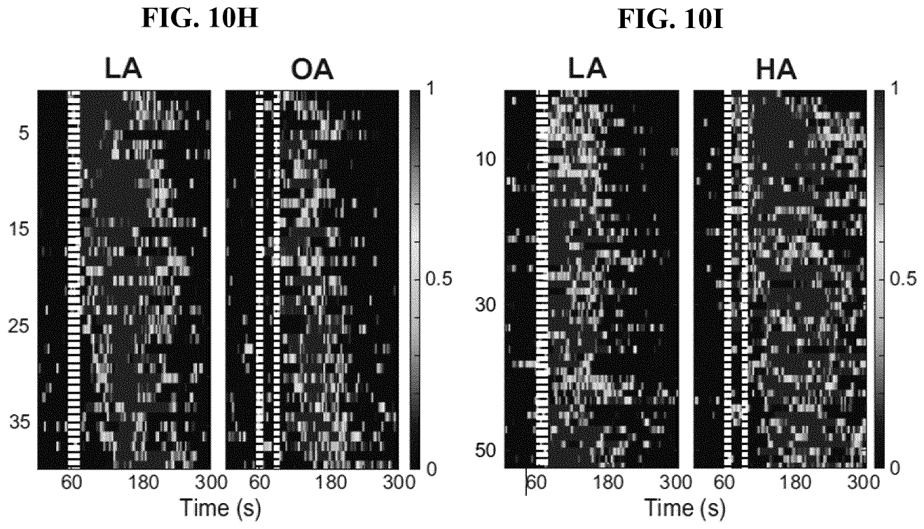

To directly examine and monitor the fat responses of vagal sensory neurons, functional imaging of the nodose ganglion (which contains the cell bodies of vagal neurons) was performed. The genetically encoded activity sensor GCaMP6s was targeted to vagal sensory neurons using Vglut2-Cre animals, and a one-photon calcium imaging set-up was used coupled to synchronous intestinal delivery of fat, sugar and other stimuli to record neuronal responses in vivo, with real time kinetics (FIG. 2E, FIG. 10A, FIG. 10B, FIG. 10C). To administer the stimuli, a catheter was placed into the duodenal bulb, and an exit port was created by transecting the intestine 10 cm distally. During each imaging session, the intestine was exposed to a pre-stimulus application of PBS, a 10 s or 60 s exposure to the test stimulus, and a 200 s post-stimulus wash; this regime was repeated at least 3 times for each stimulus. Using this preparation, intestinal infusion of fat stimuli (e.g. linoleic acid or pre-digested Intralipid), but not vehicle control, evoked robust responses in a unique subset of vagal neurons (17% of GCaMP-expressing Vglut2 neurons) (FIG. 2E, FIG. 10C, FIG. 10D); the responses were reproducible and time-locked to stimulus delivery (FIG. 2F, FIG. 10C). The same neurons responded to a wide range of middle and long chain fatty acids (FIG. 10D, FIG. 10E, FIG. 10F, FIG. 10G, FIG. 10H, FIG. 10I), thus defining a distinct subset of vagal neurons activated by intestinal fat stimuli.

Intestinal application of glucose also activates a subset of vagal neurons, and are part of the essential gut-brain axis driving the development of sugar preference. Here, how vagal neurons differentially respond to fat and sugar nutrient signals in the gut was examined.

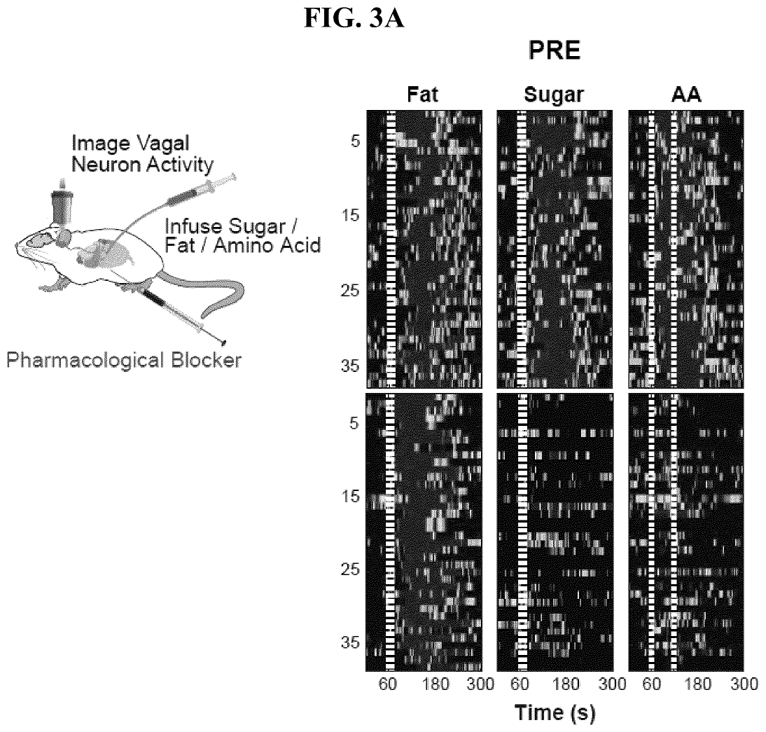

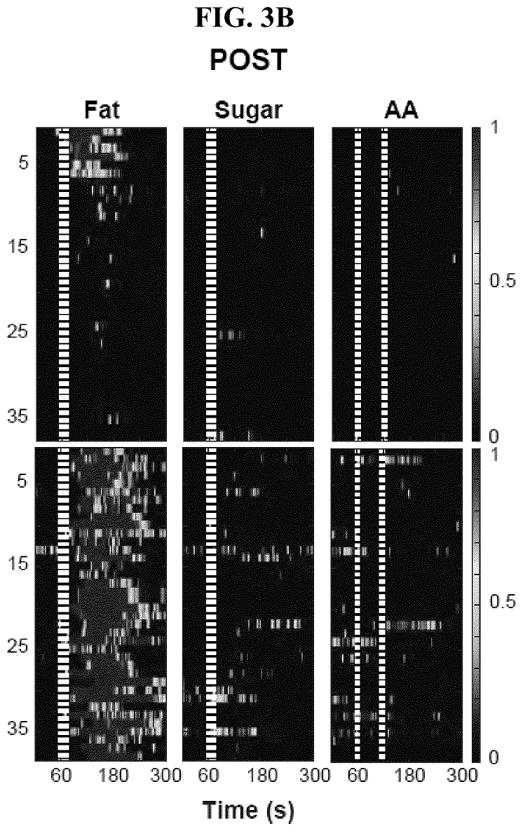

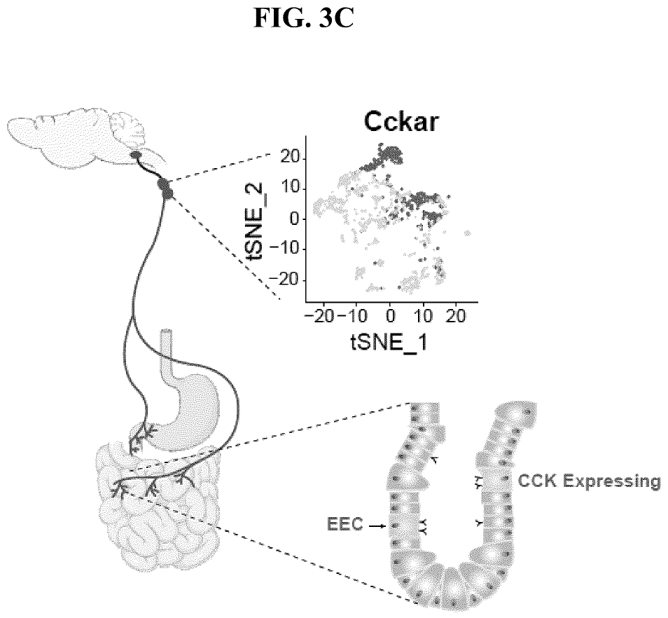

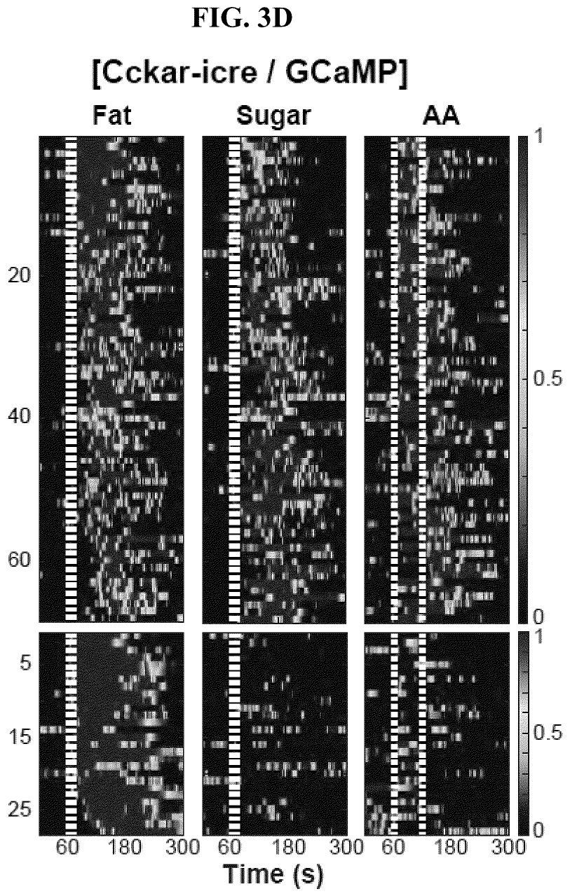

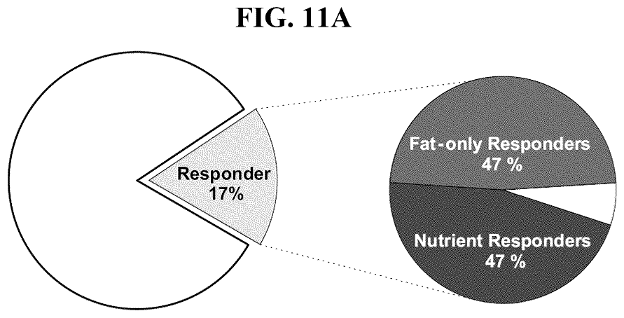

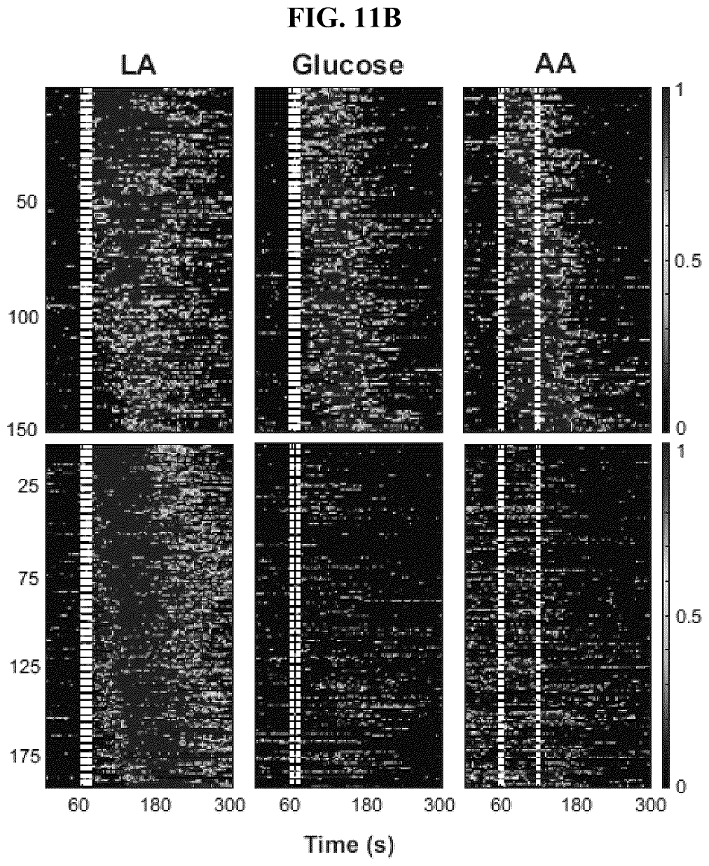

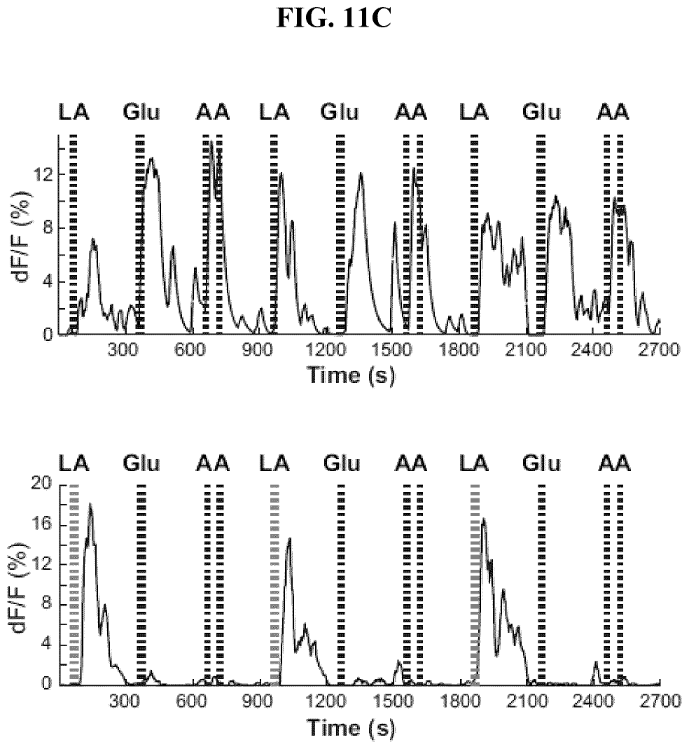

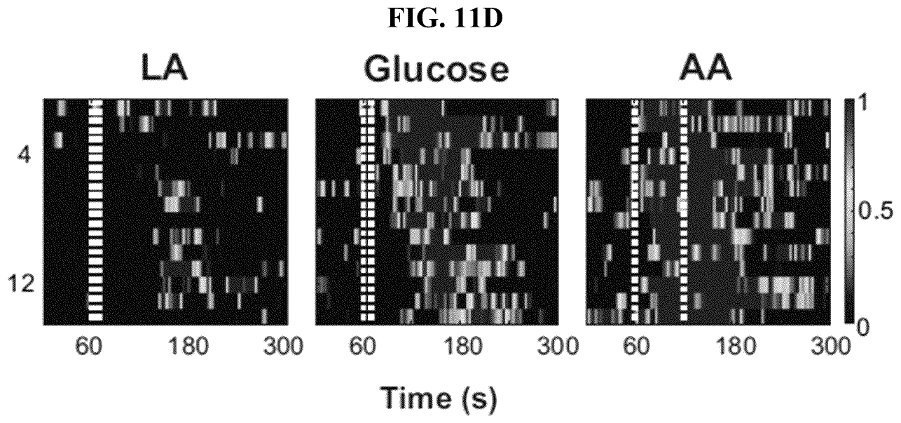

The activity of vagal neurons was recorded to alternating gut stimulation with sugar and fat. Remarkably, from over 1800 vagal sensory neurons examined from 22nodoses, two distinct groups of vagal neurons were identified: One group (˜8% of the total imaged neurons) responded to both sugar and fat. The other, a non-overlapping group (also ˜8% of the neurons), responded only to fat, but not to sugar (FIG. 2G, FIG. 11A). Notably, the subset responding to sugar and fat was also activated by amino acids (FIG. 3A, FIG. 11B, FIG. 11C). Together, these results defined two populations of vagal neurons: one, referred to herein as nutrient responders, function as sensors for all three essential macronutrients in the gut: protein, carbohydrates and fat. The other vagal population, herein referred to as fat-only, responds selectively to intestinal delivery of fat. Less than one neuron per nodose was found to respond to intestinal delivery of sugar or amino acids but not fat (FIG. 11D); however, given such small numbers, these were not considered further (it is likely that they represent sugar or nutrient responders with very small responses to fat).

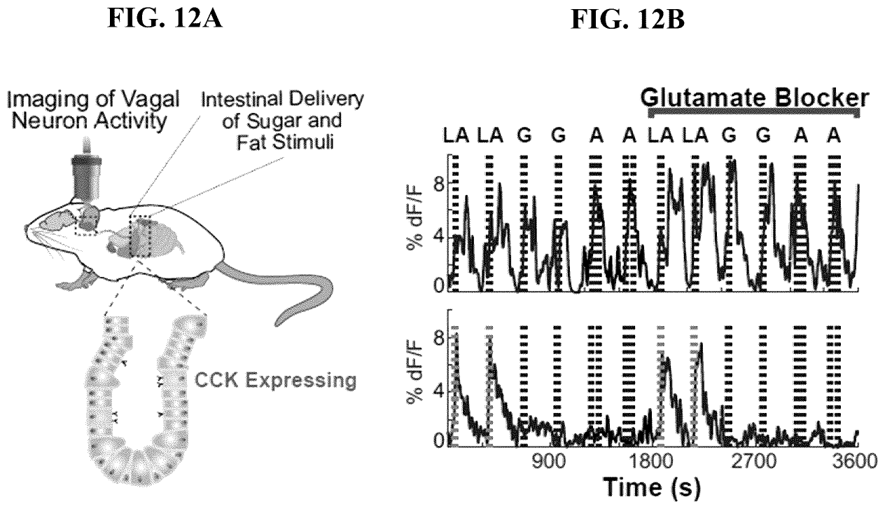

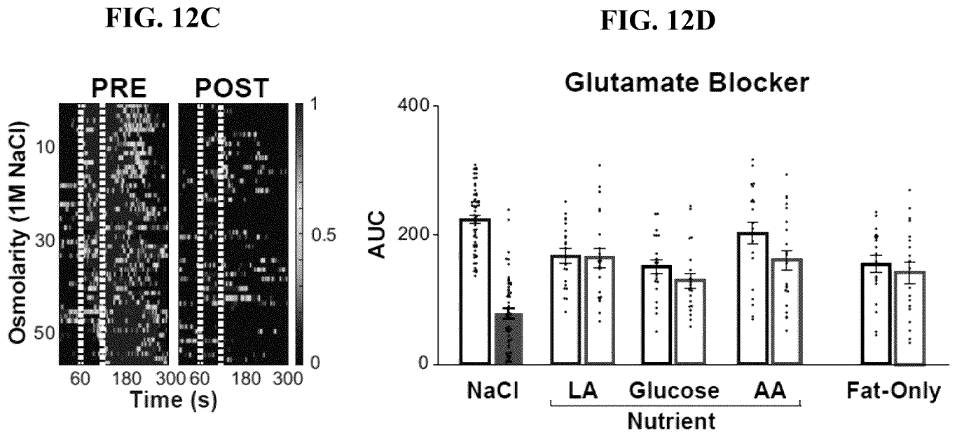

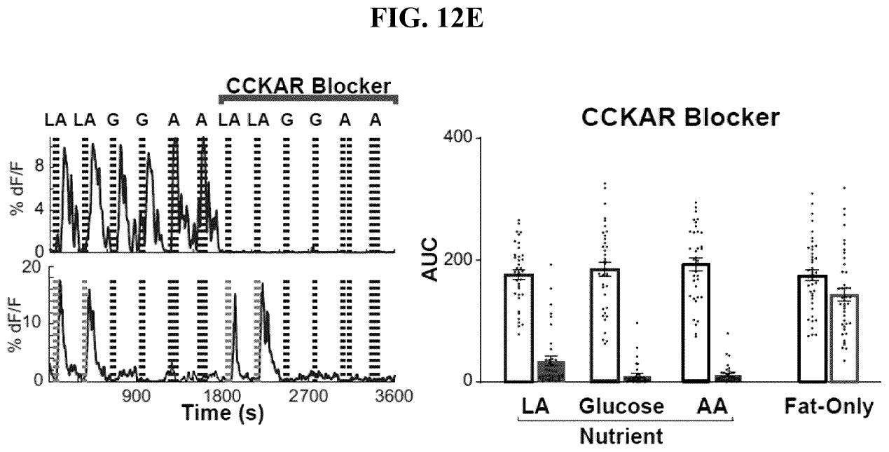

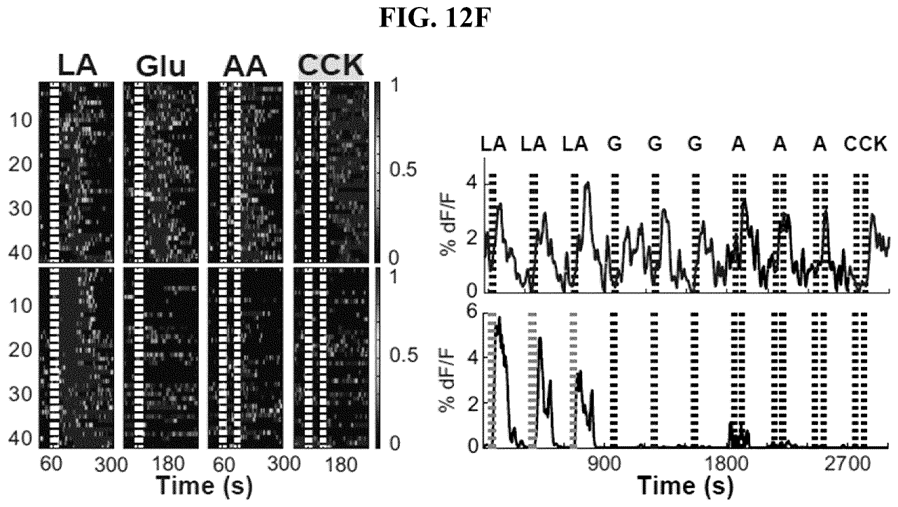

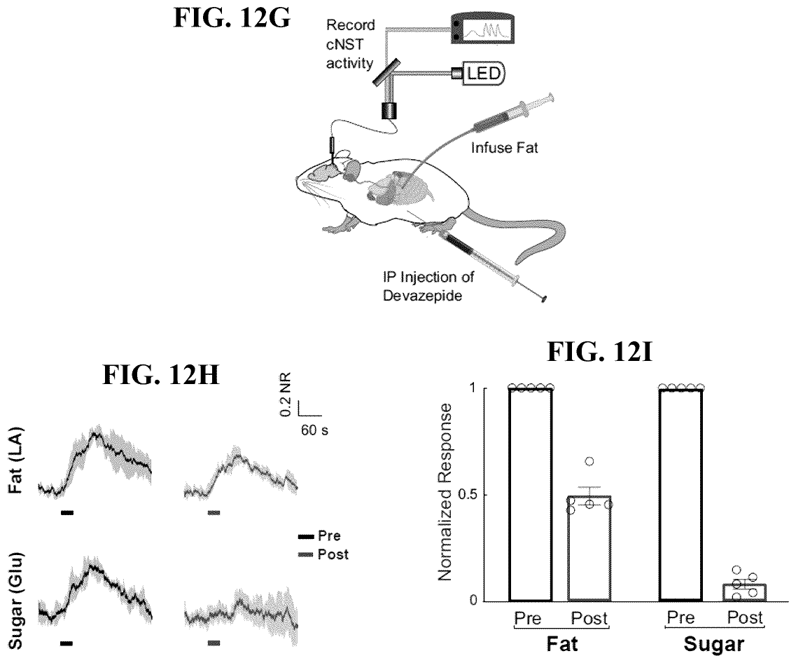

Sugar and Fat signaling in the gut First, calcium responses of vagal neurons were recorded to intestinal application of sugar, fat and amino acids, before and after addition of a mixture of AP-3 and KA glutamate receptor antagonists. Next, it was evaluated whether Cholecystokinin (CCK) is important by examining responses of vagal neurons to intestinal application of sugar, fat and amino acids, before and after pharmacologically inhibiting CCK signaling with Devazepide, a CCK-A receptor (CCKAR) antagonist (FIG. 3A). Indeed, blocking CCK signaling abolished all intestinal responses to nutrient signals (sugar, fat and amino acids). Notably, the fat-only responses remained robust and reliable (FIG. 3A, FIG. 3B, FIG. 12A, FIG. 12B, FIG. 12C, FIG. 12D, FIG. 12E, FIG. 12F, FIG. 12G, FIG. 12H, FIG. 12I). Furthermore, application of CCK peptide strongly activated the nutrient responding vagal neurons, but not the fat-only neurons (FIG. 12F). Finally, the potential role of glutamate signaling was examined by imaging responses of vagal neurons to intestinal sugar stimuli before and after addition of a mixture of 1-(+)-2-amino-3-phosphonopropionic acid (AP3) and kynurenic acid, two glutamate receptor antagonists. These results demonstrated that pharmacological inhibition of glutamate-based signaling has no effect on the gut-to-vagal sugar/nutrient-sensing circuit (FIG. 12A, FIG. 12B, FIG. 12C, FIG. 12D). Together, these results substantiate CCK as the transmitter mediating nutrient signals (sugar, amino acids and fat) in the gut-brain axis, and further distinguishes the nutrient sensing (CCK-sensitive) from the fat-only (CCK-insensitive) gut-to-brain pathways.

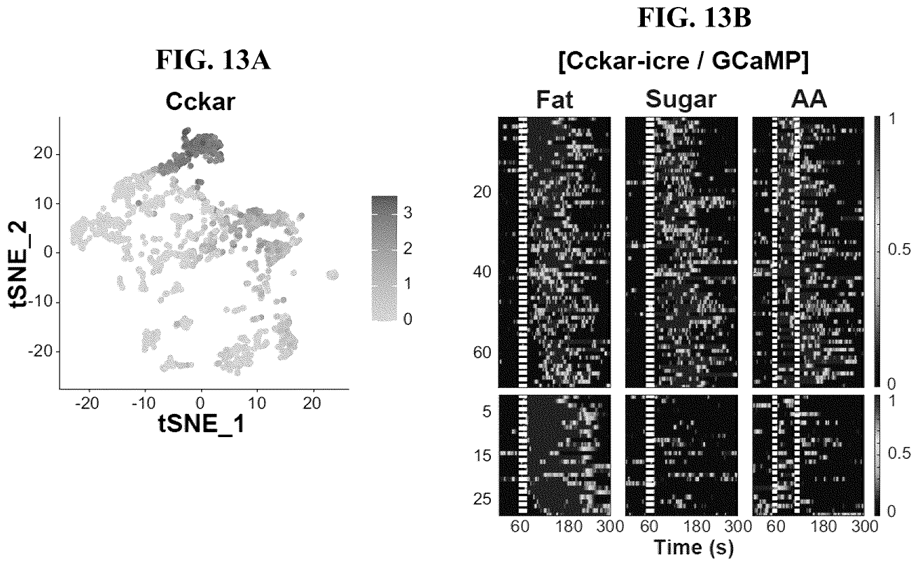

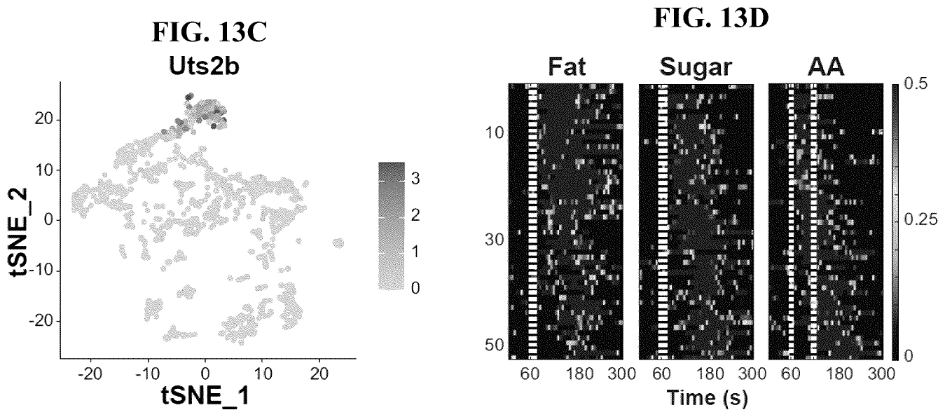

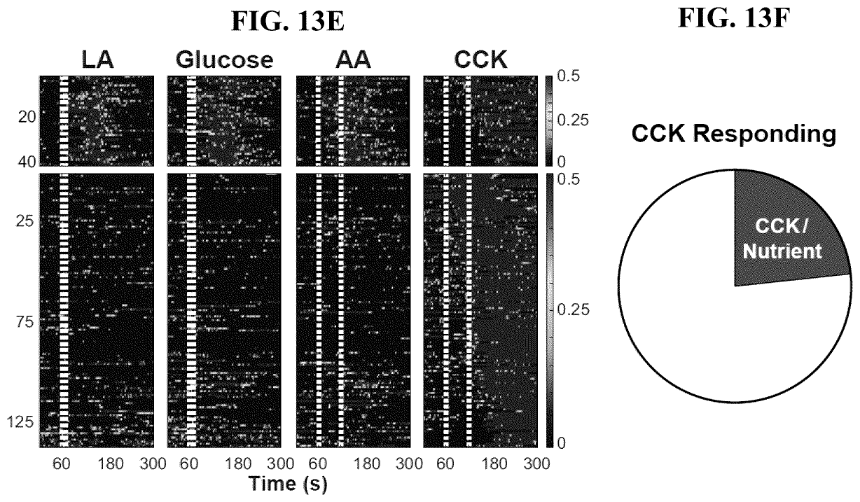

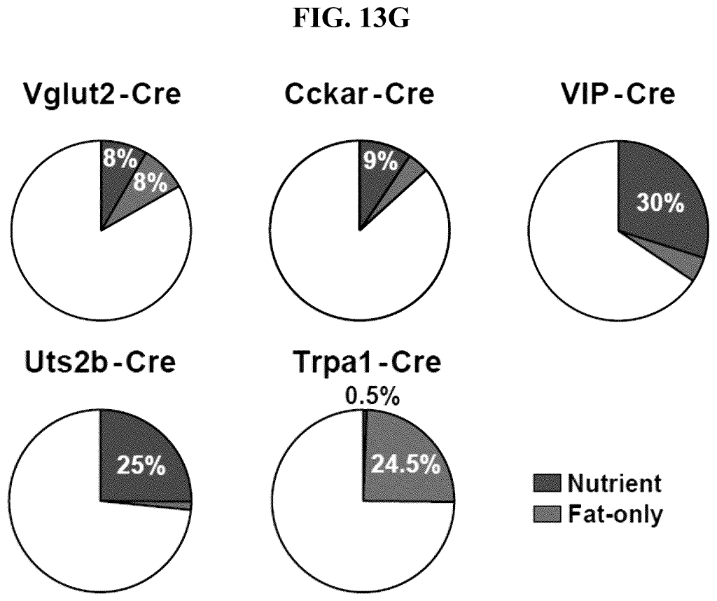

Nutrient responders in the nodose. Given that gut nutrient responders rely on CCK signaling, vagal neurons mediating this gut-to-brain signal should be defined by expression of CCK receptors (e.g., CCKAR, FIG. 3C). Therefore, Cckar-cre mice were engineered by targeting Cre recombinase to the CCK-receptor A gene, and used to functionally image the activity of GCaMP6s-expressing CCKAR-vagal neurons (FIG. 13A, FIG. 13B). Vagal CCKAR-expressing neurons exhibited robust, time-locked responses to intestinal stimulation with all 3 nutrient stimuli: fat, sugar and amino acids (FIG. 3D).

CCK is principally known as a satiety hormone, whose role is to modulate food intake by suppressing appetite. In contrast, the function of nutrient preference circuits is to promote nutrient consumption. Thus, how CCK can function both as a satiety hormone and as a nutrient preference signal was evaluated.

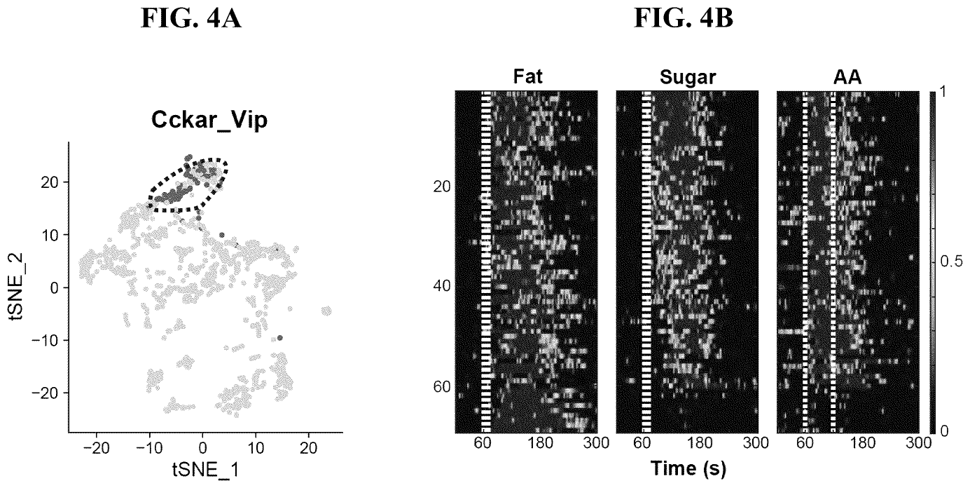

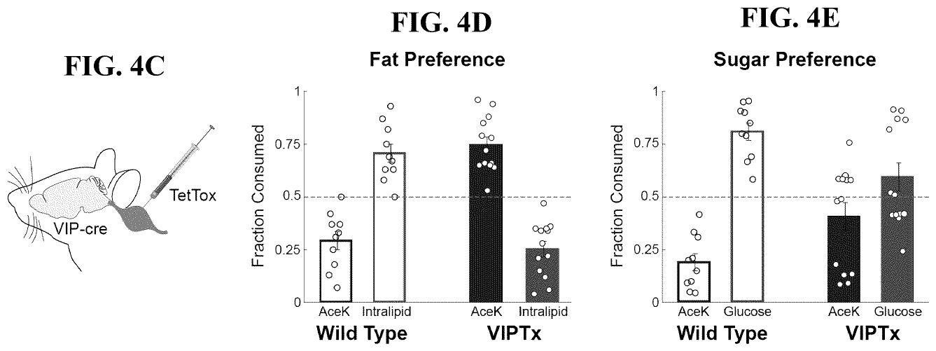

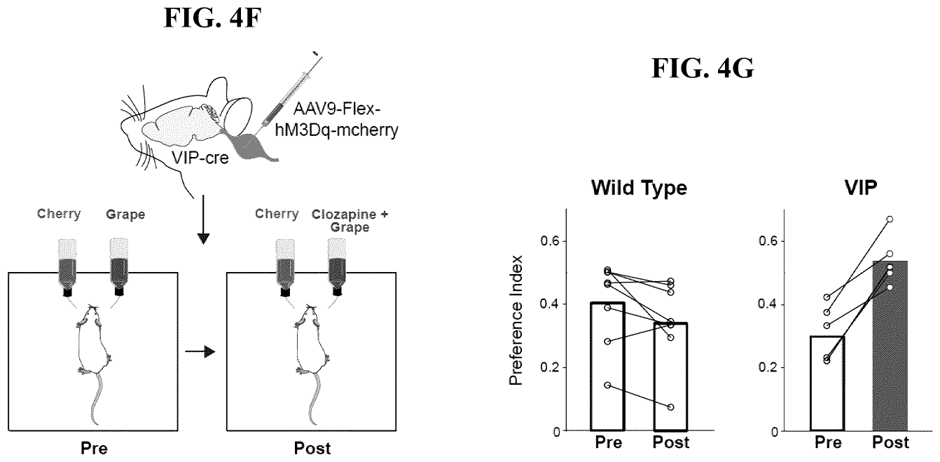

To resolve the genetic identity of the nutrient-sensing neurons, single-cell RNA seq data from the nodose ganglion was used to further resolve subsets of CCKAR-expressing neurons, and Cre driver lines expressing GCaMP6s in subsets of candidate clusters were generated. A group of CCKAR-neurons also expressing the Vasoactive Intestinal Peptide (VIP) represent the nutrient responders (FIG. 4A, FIG. 4B, FIG. 13E, FIG. 13F, FIG. 13G). This cluster was further refined by demonstrating that Urotensin 2B (Uts2b)-expressing vagal neurons, an even smaller nodose cluster overlapping CCKAR and VIP, functions as the nutrient sensors, responding to intestinal, nutrient-evoked signals, but not to fat-only stimuli (FIG. 13E, FIG. 13F, FIG. 13G). These results substantiate the segregation of the nutrient versus the fat-only circuit, and further re-define CCK in the gut not only as a satiety hormone, but also as mediator of nutrient signals.

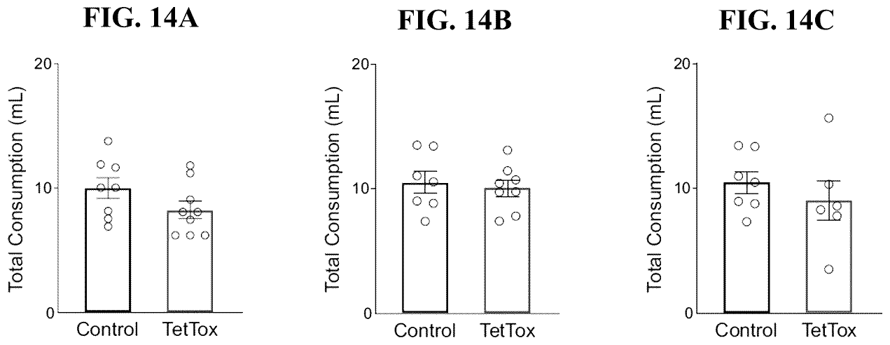

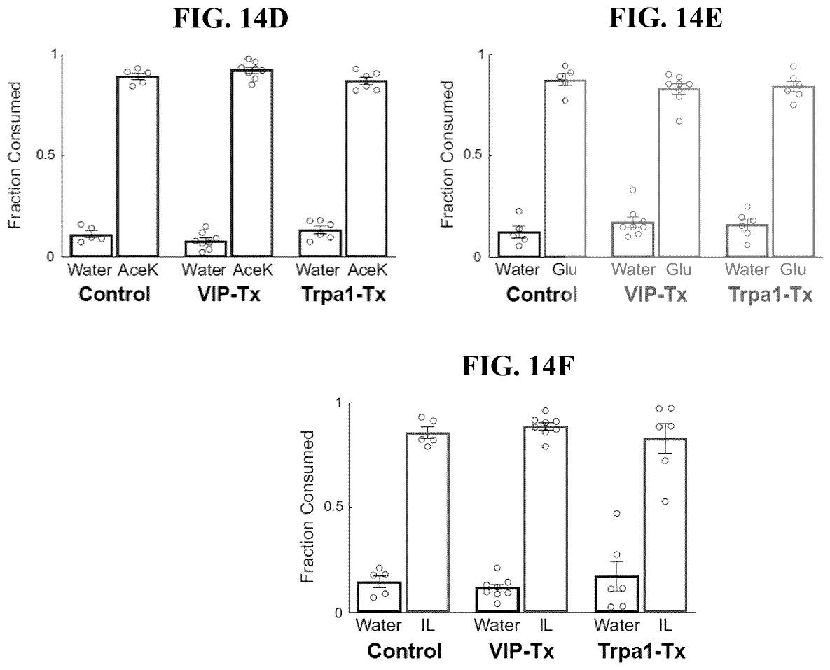

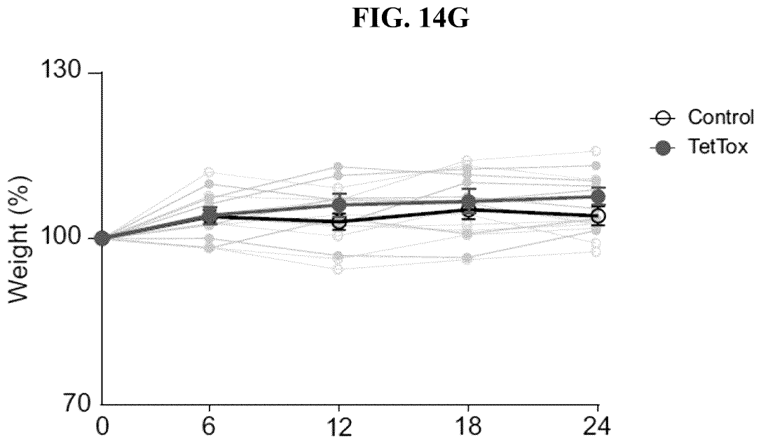

An important prediction from these results, is that inhibiting signaling from the nutrient-sensing vagal neurons should prevent the activation of the gut-brain axis, and consequently block the development of nutrient preference. Genetic silencing of the nutrient sensing vagal neurons was performed by bilaterally injecting the nodose of VIP-Cre mice with an AAV-Flex-TetTox construct (FIG. 4C, FIG. 9D). As hypothesized, blocking activity from these neurons dramatically impaired the development of nutrient preference (FIG. 4D, FIG. 4E); no changes in nutrient preference were observed in control mice injected with AAV-Flex-TetTox. The immediate, innate attraction to sugar and fat in these mice was not affected (FIG. 14A, FIG. 14B, FIG. 14C, FIG. 14D, FIG. 14E, FIG. 14F, FIG. 14G).

Finally, artificial activation of this gut-to-brain nutrient preference circuit should afford the development of new preferences, in essence driving appetitive responses to previously unpreferred stimuli. To test this proposal, the nodose of VIP-Cre mice was bilaterally injected with a Cre-dependent AAV virus encoding the excitatory designer receptor hM3Dq, such that nutrient-responding neurons could be experimentally activated by the DREADD agonist clozapine. After allowing expression of DREADD (FIG. 9E), animals were exposed to a preference assay using cherry- versus a grape-flavored solutions (FIG. 4F); to enhance consumption of these novel stimuli, both solutions were made attractive to mice by addition of an artificial sweetener. Next, a baseline preference was established for each animal (i.e., grape vs cherry), clozapine was introduced into the less-preferred flavor, and whether clozapine-mediated activation of the nutrient sensing neurons can create a new preference was determined (FIG. 4F). Indeed, after 48 h of exposure to both solutions all of the animals markedly switch their preference to the clozapine containing flavor. By contrast, mice without the designer receptor did not develop a new preference, and if anything, were slightly averse to clozapine (FIG. 4G). These results illustrate how non-natural activation of this gut-brain nutrient-sensing circuit can drive the development of a novel preference.

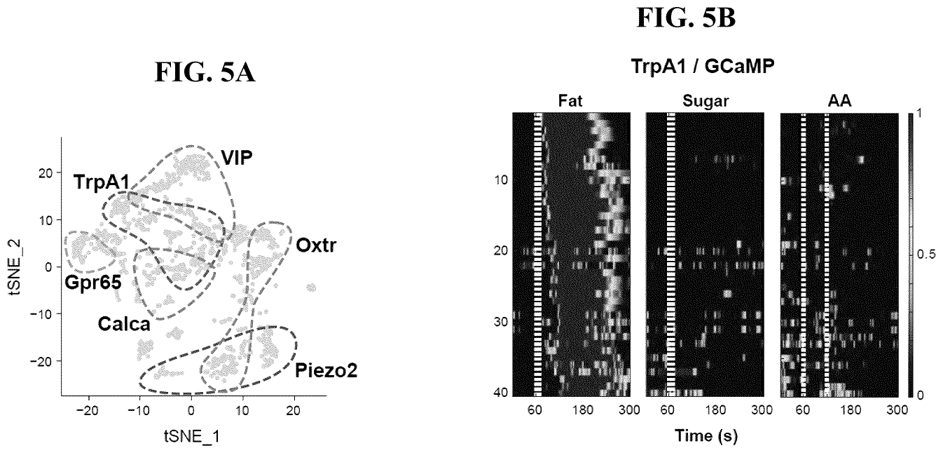

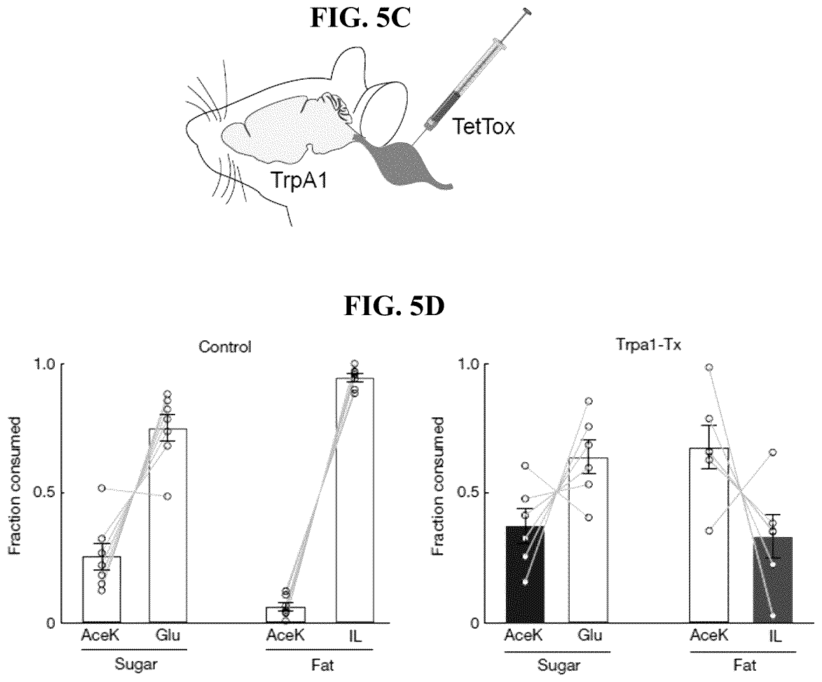

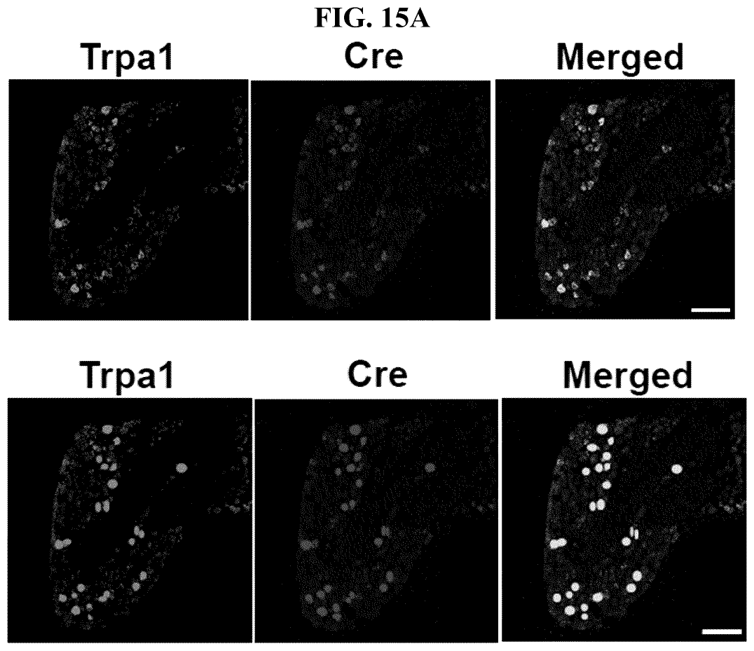

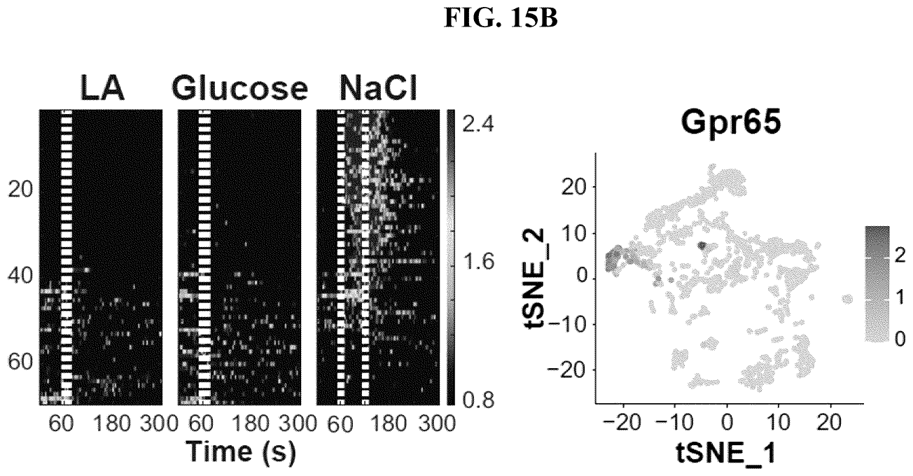

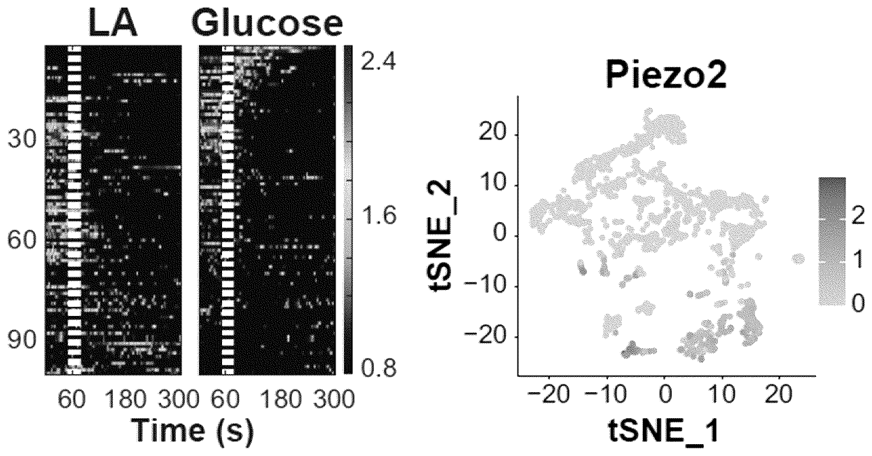

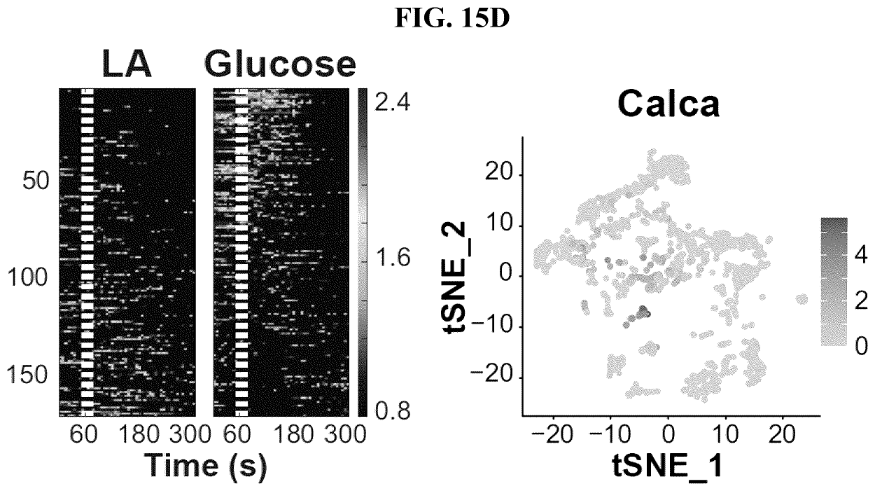

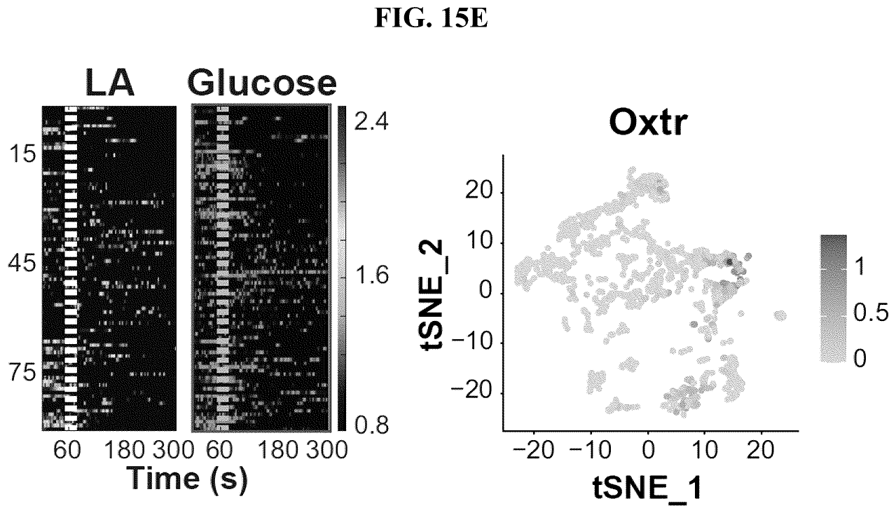

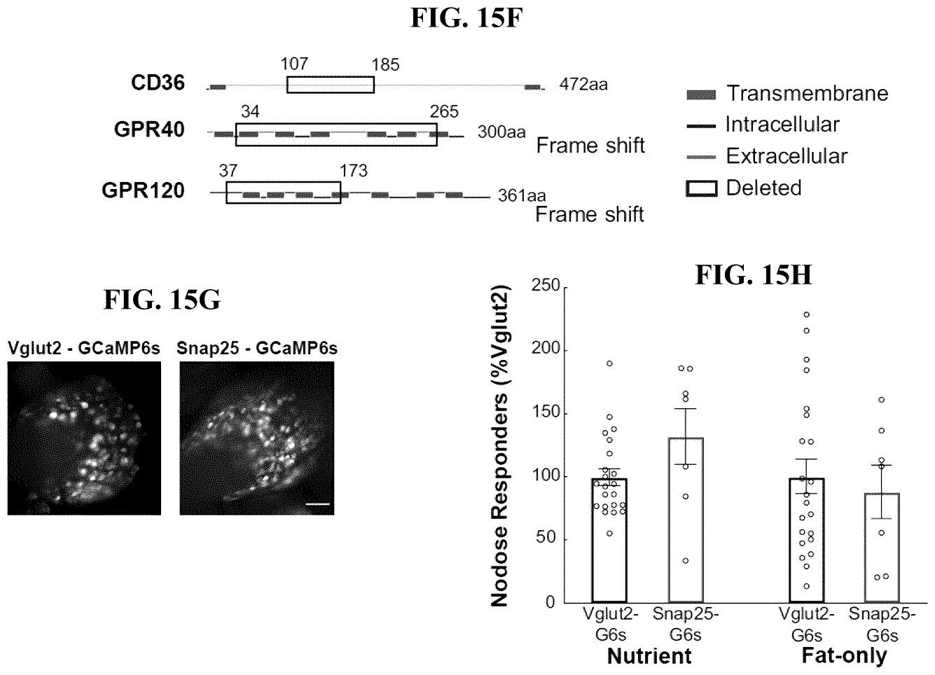

Fat-only responders in the nodose. Using the single cell RNA-seq atlas from the nodose ganglion, neurons that did not express VIP were searched (as the nutrient-sensing marker), and 5 minimally overlapping candidate clusters were identified (FIG. 5A): TrpA1, Gpr65, Piezo2, Calca, and Oxtr. Trpa1-cre mice were engineered using Crispr-cas9) (FIG. 15A), and Cre driver lines were obtained for the other 4 candidates. The results demonstrated that the TRPA1-expressing vagal cluster selectively responded to intestinal delivery of fat, but not sugar or amino acid stimuli (FIG. 5B). Vagal neurons expressing GCaMP6s in Gpr65-cre, Piezo2-cre, Calca-cre or Oxtr-cre mice were unresponsive to intestinal delivery of sugar or fat stimuli (FIG. 15B, FIG. 15C, FIG. 15D, FIG. 15E).

Genetic silencing of the fat-only circuit (i.e., TRPA1-expressing vagal neurons) should abolish the development of fat preference, but should have no impact on the development of sugar preference. Thus, the nodose of TrpA1-Cre mice was bilaterally injected with an AAV-Flex-TetTox construct, and the animals were tested for sugar-versus fat preference. Indeed, after genetic silencing, these mice can no longer develop post-ingestive preference for fat stimuli, but retain their capacity to develop post-ingestive preference for sugar (FIG. 5C, FIG. 5D). Importantly, their immediate attraction to fat is unaffected (FIG. 14A, FIG. 14B, FIG. 14C, FIG. 14D, FIG. 14E, FIG. 14F, FIG. 14G). Together, these results reveal the identity of the neurons mediating fat-only versus nutrient signals, and uncover their essential role in the gut-to-brain circuit mediating fat preference.

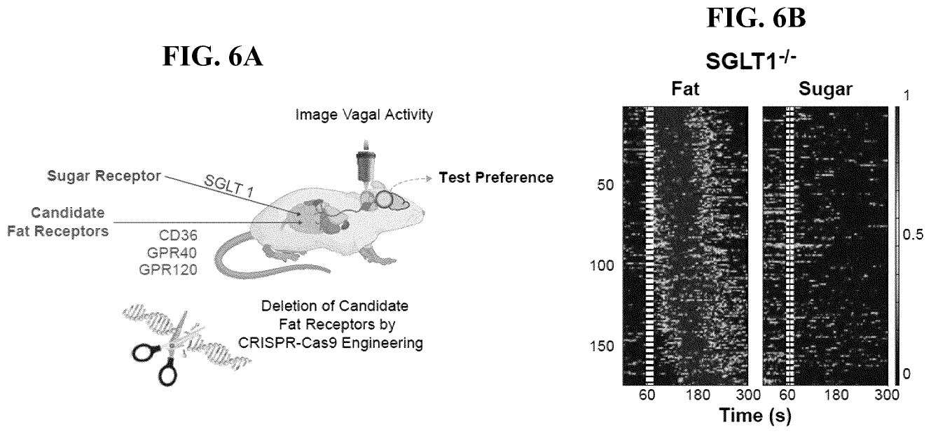

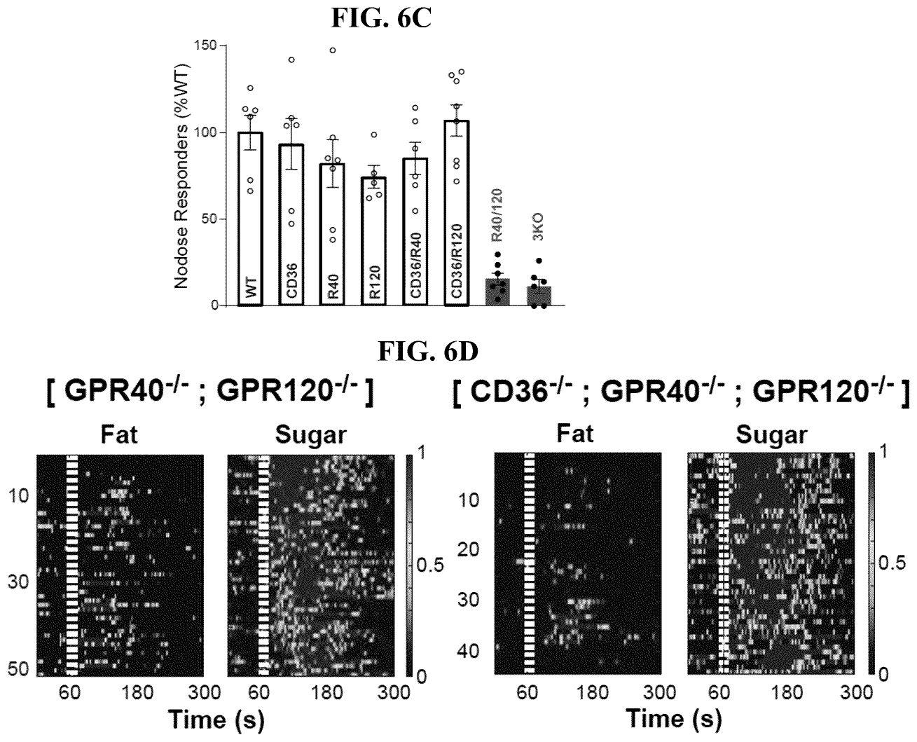

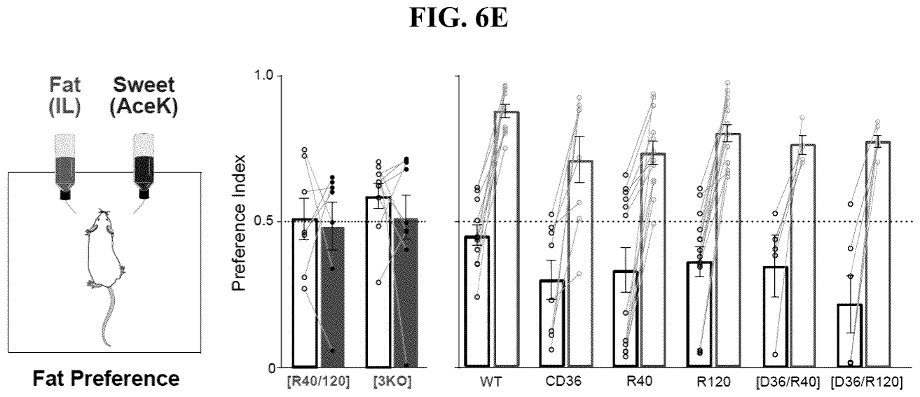

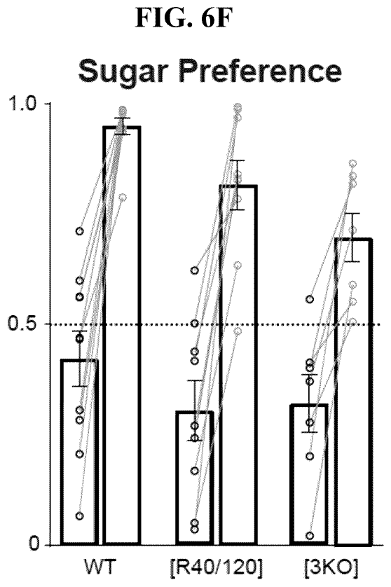

Sugar and fat sensors in the gut. Pharmacological experiments were used to demonstrate that the sodium-glucose-linked transporter-1 (SGLT1) functions as the gut receptor recognizing glucose and transmitting the gut-to-brain sugar signals (FIG. 6A). Here, SGLT1 knockout animals were generated and examined for their responses to intestinal stimulation with sugar and fat. All vagal responses to intestinal delivery of sugar are abolished (FIG. 6B). In contrast, responses to fat stimuli are unaffected.

The development of fat preference should also depend on specific fat receptors expressed on the surface of intestinal EECs. Dietary fat, once ingested and digested, is thought to be sensed by a number of putative gut receptors, including the fatty acid translocase CD36 and the G protein-coupled receptors GPR40 and GPR120. One or more of these receptors may be used by the gut-brain axis to transmit fat preference (FIG. 6A). Therefore, CRISPR-Cas9 was used to generate animals harboring knockouts of CD36, GPR40), and GPR120 in all possible knockout combinations (FIG. 15F).

Two key predictions would be that loss of the receptor(s) should abolish vagal responses to intestinal stimulation with fat, and the animals should be unable to develop fat preference in behavioral tests.

Because of the intricacies of breeding such a wide range of knockout combinations, and the need to introduce the GCaMP6s reporter for functional imaging into the various genetic backgrounds, a direct fusion of GCaMP6s to the Snap25 regulatory sequences was used rather than crossing-in a Cre-driver construct and a Cre-dependent GCaMP reporter. The Snap25-GCaMP6s construct is well expressed in nodose neurons, and compares favorably with studies using the other driver lines (FIG. 15G, FIG. 15H).

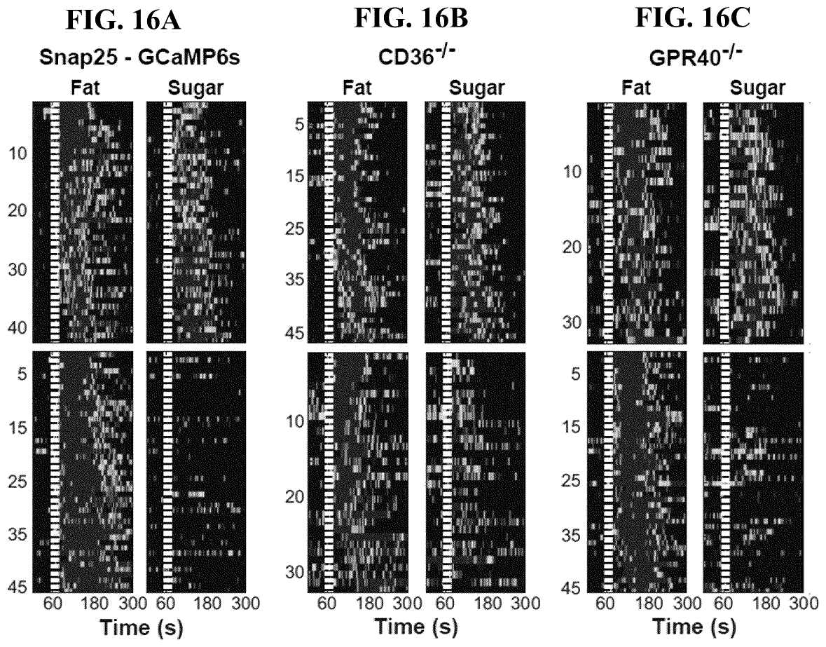

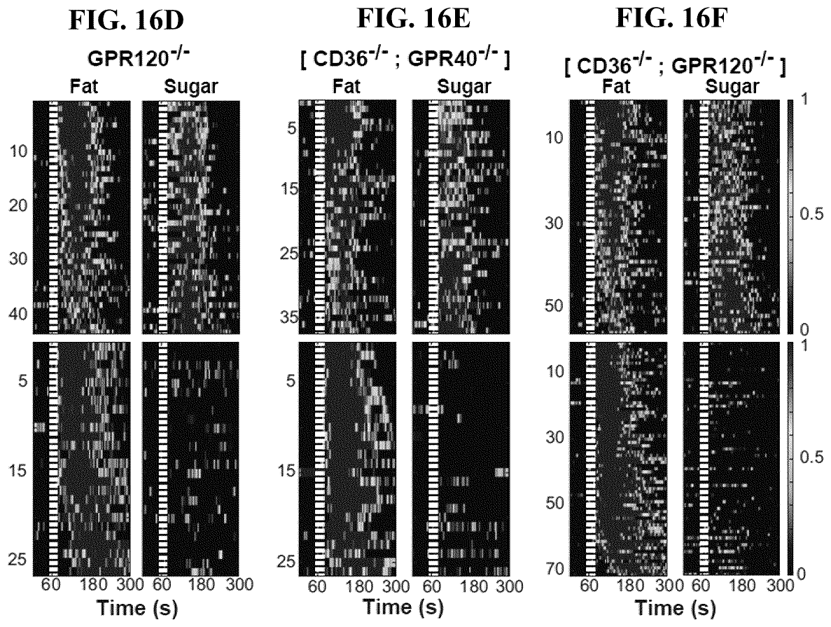

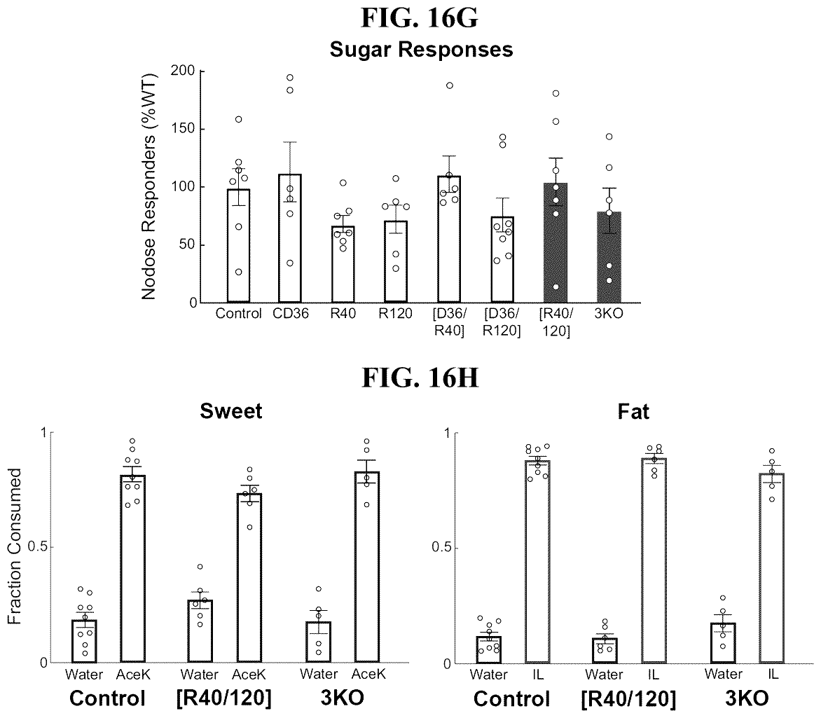

After testing all the fat receptor-deletion combinations, GPR40) and GPR120 were found to be the essential mediators of intestinal fat signals (FIG. 6C, FIG. 6D, FIG. 16A, FIG. 16B, FIG. 16C, FIG. 16D, FIG. 16E, FIG. 16F). As expected, sugar responses were unaffected in all of the mutant combinations (FIG. 16A, FIG. 16B, FIG. 16C, FIG. 16D, FIG. 16E, FIG. 16F, FIG. 16G). Both the fat-only and the fat responses of the nutrient sensing vagal neurons were abolished in the knockout animals, demonstrating that the same receptors are used in both signaling pathways (CCK-independent and CCK-dependent, respectively)

A prediction from these imaging results is that GPR40/GPR120) double knockout animals (as well as the triple knockout) should fail to develop preference for fat, while the various single and other double mutants should be unaffected. Indeed, GPR40), GPR120 and CD36 single mutants, as well as GPR40/CD36 and GPR120/CD36 double mutants were indistinguishable from control wild type mice. By contrast, the GPR40/GPR120 double (and the triple mutants) were no longer capable of developing a behavioral preference for fat (FIG. 6E). Importantly, immediate responses to fat stimuli was unaffected in the GPR40/GPR120 double mutants, further validating the fundamental difference between the taste and the gut-brain pathways (FIG. 16H). Just like control mice, fat receptor knockouts developed normal preference for sugar (FIG. 6F). Together, these results substantiate GPR40) and GPR120 as the gut sensors signaling behavioral preference for fat stimuli via the gut-brain axis.

Sugar and fat are essential nutrients, and it would be expected that dedicated circuits motivate their consumption. In addition to the taste system, these nutrients rely on a dedicated gut-to-brain post-ingestive system to detect and report the presence of intestinal sugar and fat to the brain.

Here, the fundamental role of these circuits was validated by demonstrating that genetic or pharmacological blockage of sugar or fat gut-to-brain signals, at any of the 4 stages following ingestion, abolished the development of nutrient preference: (1) by preventing binding to the sugar or fat receptors on the EEC cells, (2) by blocking the activated EEC cells from signaling to the vagal neurons, (3) by silencing the sugar-or fat-activated vagal neurons, and preventing them from signaling the brain, and (4) finally, by preventing the cNST neurons receiving the gut-brain signal from broadcasting the presence of intestinal nutrients to the rest of the brain.

An unexpected finding from these studies was the discovery of a single gut-to-brain pathway, based on CCK signaling, that functions as a generalist detector informing the brain of the intestinal presence of any of the three essential nutrients: sugar, amino acids and fat. Although each nutrient uses its own dedicated receptors in the gut, the convergence of the signal into a unique class of vagal neurons highlights the simple and elegant logic of this circuit: after the gut cells are activated, the circuit does not need to preserve the identity of the specific nutrient stimulus, only to ensure that the emerging gut-brain signal motivates consumption.

Given that CCK functions as the signaling molecule in the gut for the general nutrient sensing pathway, there should be a subset of CCK-positive EECs that co-express the sugar (SGLT1) and the fat (GPR40) and GPR120) preference receptors. Indeed, examination of single cell RNA atlases from both rodent and human gut tissue proved this to be the case. These data uncovered a select population of vagal, CCK-responding neurons (marked by the expression both of CCKAR and UTS2b), that is separate from the larger population of CCK-receptor expressing, and function as the vagal partners of the CCK-releasing intestinal nutrient sensing enteroendocrine cells. These neurons act as the principal conduit in the gut-brain axis transmitting nutrient signals from the intestines to the brain.

Intestinal fat sensing uses its own additional dedicated line via the gut-brain axis. Interestingly, genetic silencing of fat signals either via the nutrient or the fat-only pathway abolished the development of fat preference, indicating that these two are not redundant pathways, but rather that both play an important role.

### Methods

Animals. All procedures were carried out in accordance with the US National Institutes of Health (NIH) guidelines for the care and use of laboratory animals, and were approved by the Institutional Animal Care and Use Committee at Columbia University. Adult mice older than 6 weeks of age and from both sexes were used in all experiments. C57BL/6J (JAX 000664), TRAP2 (JAX 030323), TRPM5 KO (JAX 013068), Ai96 (JAX 028866), Ai162 (JAX 031562), Vglut2-IRES-cre (JAX 028863), Gpr65-IRES-cre (JAX 029282), Vip-IRES-cre (JAX 010908); Uts2b-cre (JAX 035452); Piezo2-cre (JAX 027719); Oxtr-cre (JAX 031303); Calca-cre (JAX 033168); Snap25-2A-GCaMP6s (JAX 025111) and Penk-IRES2-cre (JAX 025112).

Generation of genetically modified mice. To engineer Trpa1-IRES-cre knock-in mice51, a single guide RNA (sgRNA) (targeting CACAGAACTAAAAGTCCGGG (SEQ ID NO: 1)) was selected to introduce an IRES-cre construct immediately downstream of the endogenous Trpa1 stop codon. A single-stranded DNA donor containing gene-specific homology arms (150 bp each) and the IRES-cre fragment (Addgene #61574) was generated using the Guide-it Long ssDNA Production System (Takara Bio). Cas9 protein (100 ng/μl), sgRNA (20 ng/μl) and ssDNA donor (10 ng/μl) were co-injected into the pronuclei of fertilized zygotes from B6CBAF1/J parents. Founder pups were screened for the presence of the knock-in allele using PCR, and candidates were validated by Sanger sequencing. SGLT1-knockout mice were generated by co-injecting Cas9 mRNA (100 ng/μl) with sgRNA (50 ng/μl) targeting CGCATTGCGAATGCGCTCGT (SEQ ID NO: 2), resulting in a frameshift after the 20th residue and early termination after the 27th residue (wild-type SGLT1 is a 665-amino-acid protein). Homozygous SGLT1-knockout mice were bred and maintained on fructose-based rodent diet with no sucrose or cornstarch (Research Diets #D08040105). The mutant allele was validated by DNA sequencing. To generate knockout mice for fat receptors (CD36, GPR40 and GPR120), Cas9 protein (50 ng/μl) was co-injected with a total of 6 sgRNAs (7 ng/μl each: CD36: AAATATAACTCAGGACCCCG (SEQ ID NO: 3) and TAGGATATGGAACCAAACTG (SEQ ID NO: 4); GPR40: AGTGAGTCGCAGTTTAGCGT (SEQ ID NO: 5) and GAAGTTAGGACTCATCACAG (SEQ ID NO: 6); GPR120: CGACGCTCAACACCAACCGG (SEQ ID NO: 7) and ACGCGGAACAAGATGCAGAG (SEQ ID NO: 8)). The founder mice were validated by DNA sequencing and used to generate various homozygous knockout mice (that is, single, double and triple knockouts). All mutations in the individual homozygous lines were validated by DNA sequencing. To engineer transgenic mice expressing Cre recombinase from the Cckar gene (Cckar-cre mice), a cre cassette was introduced at the ATG start codon of the Cckar gene using a 151 kb bacterial artificial chromosome (BAC) (RP23-50P5) carrying the Cckar gene, as described previously (Lee, H., Macpherson, L. J., Parada, C. A., Zuker, C. S. & Ryba, N. J. P. Rewiring the taste system. Nature 548, 330-333 (2017).

Fos stimulation and histology. Stimuli consisted of 20% Intralipid (sc215182, Santa Cruz Biotechnology), 10% linoleic acid, 10% oleic acid, 0.3% xanthan gum or 10% mineral oil. Stimuli were emulsified by dilution into milliQ water containing 0.1% xanthan gum and 0.05% Tween 80, and vortexed for a minimum of 10 min. Note that high concentration of Intralipid for Fos and TRAP2-labelling experiments was used to ensure enough Intralipid is consumed and digested during the 90 min stimulation window. By contrast, when performing 48 h behavioral tests examining the development of fat preference, a lower concentration of 1.5% Intralipid was used, particularly to ensure that the fat and the AceK (3 mM) are similarly attractive. To motivate drinking behavior during the 90 min Fos induction experiments, C57BL/6J mice were water-restricted for 23 h, given access to 1 ml of water for 1 h, and then water-restricted again for another 23 h. Previously, such water restriction prior to the 90 min drinking test did not affect the selectivity of cNST labelling (for example, no labelling in response to water or AceK; see also FIG. 8A, FIG. 8B, FIG. 8C, and FIG. 8D, FIG. 8E, FIG. 8F, FIG. 8G, FIG. 8H, FIG. 8I, FIG. 8J, FIG. 8K, FIG. 8L). Mice had the full complement of food during water restriction (this is essential during Fos labelling experiments as food restriction would activate a wide range of additional circuits, including food-reward circuits upon presentation of sugar or fat stimuli). All Fos experiments consisted of 90 min of exposure to the stimuli; mice were housed individually and all the nesting material and food was removed from their cages. After 90 min, mice were perfused transcardially with PBS followed by 4% paraformaldehyde. Brains were dissected and fixed overnight in paraformaldehyde at 4° C. The brains were sectioned coronally at 100 μm and labelled with anti-c-Fos (SYSY, no. 226004 guinea pig, 1: 5,000) diluted in 1×PBS with 5% normal donkey serum (EMD Millipore, Jackson ImmunoResearch) and 0.3% Triton X-100 for 48 h at 4° C., and then Alexa Fluor 647-conjugated donkey anti-guinea pig (Jackson ImmunoResearch) for 24 h at 4° C. Images were acquired using an Olympus FluoView 1000 confocal microscope. Quantification of Fos labelling was carried out by recording the number of positive neurons in an equivalent 200×200 um area of the cNST (bregma—7.5 mm) and area postrema. For intragastric stimulation, the catheter was placed as previously described (Tan, H. E., et al., The gut-brain axis mediates sugar preference. Nature 580, 511-516 (2020); Ueno, A., et al., Mouse intragastric infusion (iG) model. Nat. Protoc. 7, 771-781 (2012)). Mice were individually housed and allowed to recover for at least five days before stimulus delivery. A syringe pump microcontroller (Harvard Apparatus) was used to deliver 1.5 ml of the control PBS or 20% Intralipid solution4 at 0.050 ml/min.

Two-bottle preference assays. No behavioral experiments, including the short-term assays for taste responses, or the 48 h tests examining the development of sugar or fat preference used water-restricted or food-deprived mice. Mice were given ad libitum access to food and water for several days prior to the behavioral tests; any food or water restriction would severely affect the mice's behavior in preference or taste responses. Development of fat preference: mice were first tested for their initial preference between 1.5% Intralipid and 3 mM AceK (pre testing) by completing 100 drinking trials. Each trial was initiated by the first lick and lasted for 5 s; the drinking ports then re-opened after 30 s of inter-trial interval. Next, mice were exposed to 500 licks to both 1.5% Intralipid and 3 mM AceK; this was repeated twice. Mice were then tested for the development of fat preference over 36 h using the 5 s trials. The pre and post-preference indexes were calculated by dividing the number of licks to fat by the total lick count during the first 2-4 h (100 trials) of baseline measurements (pre) and during the last 2-4 h (100 trials) of the behavioural session (post), respectively. In order to perform the two-bottle preference assay using large numbers of mice, the setup was modified by using an LCD-based lick counter. The ‘pre’ preference index was calculated as the number of licks to fat divided by the total lick count during the first 4 h; the “post” preference index was calculated as the number of licks to fat divided by the total lick count during the last 4 h of the session. Mice had ad libitum access to food throughout. The mice with a pre index>0.75 were not used owing to their high initial preference for fat (less than 20% of total tested mice had to be eliminated due to this strong bias).

Fat, sugar and amino acid intestinal stimulation. Stimuli for nodose imaging experiments were as follows. Sugar: 500 mM glucose. Amino acids: a mix consisting of 50 mM methionine, 50 mM serine, 50 mM alanine, 50 mM glutamine and 50 mM cysteine dissolved in PBS. Fat: 10% linoleic acid, 10% linolenic acid, 10% hexanoic acid, 10% DHA, 10% oleic acid, diluted in PBS containing 0.1% xanthan gum and 0.05% Tween 80, and vortexed for a minimum of 10 min. Vehicle control: 0.1% xanthan gum and 0.05% Tween 80. For sugar and fat intestinal stimulation in imaging experiments, a 10 s window of stimulation was used; for amino acids, a 60 s stimulus was used, as lower concentrations of each in the mix of several amino acids was used (see above). Note that if using Intralipid mix (a 20% soy bean oil emulsion, Santa Cruz) for nodose imaging experiments (rather than consumption where it would be naturally digested and broken down into short, medium and long chain fatty acids), the material was pre-digested with lipases (mimicking its natural course of action upon ingestion). Using undigested complex oils for intestinal stimulation in imaging experiments yielded inaccurate and unreliable responses (data not shown). Intralipid was incubated with 4 mg/ml lipase (sigma) in PBS plus 10 mM CaCl2 for a minimum of 5 h at 37° C.

Stereotaxic surgery. Mice were anaesthetized with ketamine and xylazine (100 mg/kg and 10 mg/kg, intraperitoneal), and placed into a stereotaxic frame with a closed-loop heating system to maintain body temperature. The coordinates (Paxinos stereotaxic coordinates) used to inject and place recording fibres in the cNST were: caudal 7.5 mm, lateral ±0.3 mm, ventral 3.7-4 mm, all relative to Bregma. The fibre photometry experiments used a 400 μm core, 0.48 NA optical fibre (Doric Lenses) implanted 50-100 μm over the left cNST. TRAP2 mice were stereotaxically injected bilaterally in the cNST with AAV9-Syn-DIO-mCherry (300 nl per mouse), AAV9 DIO eGFP-RPL10a (300 nl per mouse) or AAV9 CBA.FLEX-TetTox54 (300 nl per mouse).

Genetic access to fat preference neurons in the brain. The TRAP strategy was used in TRAP220,55 mice to gain genetic access to fat-activated neurons in the cNST. A minimum of 5 days after injection, the AAV-injected TRAP2 mice or TRAP2; Ai9 mice were water-restricted for 23 h, given access to 1 ml of water for 1 h, water-restricted again for another 23 h (with ad libitum food), and then presented with 20% Intralipid ad libitum in the absence of food and nesting material. After 1 h, mice were injected intraperitoneally with 12.5 mg/kg 4-hydroxytamoxifen (Sigma H6278) and placed back in the same cage for an additional 3 h. Following 4 h of Intralipid exposure, mice were returned to regular home-cage conditions (group-caged, with nesting material, ad libitum food and water). Mice were used for experiments a minimum of 10 days after this TRAP protocol. C57BL/6J and TRAP2 mice expressing TetTox in the cNST were tested in the two-bottle Intralipid versus sweetener preference assay for 48 h, as described previously (Tan, H. E., et al., The gut-brain axis mediates sugar preference. Nature 580, 511-516 (2020)). Note that mice were never food-deprived prior to TRAPping, so as to prevent unrelated labelling and confounds from the activation of feeding and food-reward responding neurons.

Fibre photometry. Vglut2-cre; Ai96 mice were placed in a stereotaxic frame and implanted with a 400 μm core, 0.48 NA optical fibre (Doric Lenses) 50-100 μm over the left cNST. Photometry experiments were conducted as described previously (Tan, H. E,. et al., The gut-brain axis mediates sugar preference. Nature 580, 511-516 (2020); Lerner, T. N., et al., Intact-brain analyses reveal distinct information carried by SNc dopamine subcircuits. Cell 162, 635-647 (2015)). To quantify the effects of vagotomy, the ratio of stimulus-related peak amplitude of the normalized trace was calculated (within 120 s of stimulus onset) prior vagotomy versus after vagotomy. The duodenal catheterization surgery was carried out as described previously (Tan, H. E., et al., The gut-brain axis mediates sugar preference. Nature 580, 511-516 (2020)). Stimulus delivery was performed via a series of peristaltic pumps (BioChem Fluidics) operated via custom Matlab software and Arduino microcontroller. Stimuli and washes were delivered through separate lines that converged on a common perfusion manifold (Warner Instruments) connected to the duodenal catheter. Trials consisted of a 60-s baseline (PBS 200 μl/min), a 30 s stimulus (200 μl/min), and a 3-min washout period (150 s at 600 μl/min, and 30 s at 150 μl/min). Stimuli were each presented three times in an interleaved fashion. The vagotomy procedure was carried out after the first round of stimulus as described previously (Tan, H. E., et al., The gut-brain axis mediates sugar preference. Nature 580, 511-516 (2020); Allen, I.C., in Mouse Models of Allergic Disease, Methods and Protocols Vol. 1032 (ed. Allen, I. C.) v-vi (Humana Press, 2013)).

Nodose ganglion injection experiments. Genetic vagal silencing experiments. Cre-expressing mice (Vip-cre and Trpa1-cre) were anaesthetized with ketamine and xylazine (100 mg/kg and 10 mg/kg, intraperitoneal). The skin under the neck was shaved and betadine and alcohol were used to scrub the skin three times. A midline incision (˜1.5 cm) was made and the trachea and surrounding muscles were gently retracted to expose the nodose ganglia. AAV9 CBA.FLEX-TetTox (600 nl per ganglion) containing Fast Green (Sigma, F7252-5G) was injected in both left and right ganglia using a 30° beveled glass pipette (custom-beveled Clunbury Scientific). At the end of surgery, the skin incision was closed using 5-0) absorbable sutures (CP medical, 421A). Mice were allowed to recover for a minimum of 26 days before 2-bottle preference tests for sugar and fat. Almost all of the Vip-cre mice survived the surgical procedure and bilateral injections, whereas only 50% of the Trpa1-cre mice survived. The Trpa1-cre knock-in line was validated by in situ hybridization experiments (FIG. 15A). Fixed frozen nodose ganglia were sectioned at 16 μm thickness and processed for mRNA detection using the RNAscope Fluorescent Multiplex Kit (Advanced Cell Diagnostics) following the manufacturer's instructions. The following RNAscope probes were used: Trpa1 (catalogue no. 400211-C3) and Cre-04 (catalogue no. 546951).

Chemogenetic activation experiments. For gain-of-preference experiments, Vip-cre mice were injected bilaterally with 600 nl per ganglion of an AAV carrying the Cre-dependent activator DREADD (AAV9-Syn-DIO-hM3Dq-mCherry) and were allowed to recover for a minimum of three weeks before behavioral tests. Control and Vip-cre mice were tested in a two-bottle grape versus cherry flavor-preference assay (grape: 0.39 g/l Kool-Aid Unsweetened Grape, cherry: 0.36 g/l Kool-Aid Unsweetened Cherry, both containing 1 mM AceK). Flavor-preference tests were carried out as previously described (Tan, H. E. et al. The gut-brain axis mediates sugar preference. Nature 580, 511-516 (2020)).

Vagal calcium imaging. Each mouse was anaesthetized with ketamine (100 mg/kg) and xylazine (10 mg/kg). The mice were tracheotomized, and the nodose ganglion was exposed for imaging exactly as previously described (Tan, H. E., et al., The gut-brain axis mediates sugar preference. Nature 580, 511-516 (2020)). For CCKAR blocker experiments, devazepide (Sigma) was dissolved in DMSO and diluted to a final dose of 4 mg/kg in saline. For glutamate receptor blocker experiments, a mixture of metabotropic glutamate receptor antagonist AP3 (2 mg/kg) and ionotropic glutamate receptor antagonist kynurenic acid (300 μg/kg) was used. CCKAR and glutamate receptor blockers were delivered both into the intestines and abdominal cavity; after a 5 min incubation period, the imaging session was started. For CCK application, the intestines, still attached to the anaesthetized mouse, were partly placed on a 25 mm petri dish to allow delivery (60 s) and washout (>180 s) of the stimuli (1 μg/ml CCK peptide; Bachem 4033101). Note that for nodose imaging experiments using sugar, glucose stimuli consisted of 10 s pulses since stimulating with high concentration (>250 mM) for long pulses (60 s or more) strongly activates nutrient-independent vagal responses ((Tan, H. E., et al., The gut-brain axis mediates sugar preference. Nature 580, 511-516 (2020); Williams, E. K., et al., Sensory neurons that detect stretch and nutrients in the digestive system. Cell 166, 209-221 (2016); Ichiki, T., et al., Sensory representation and detection mechanisms of gut osmolality change. Nature 602, 468-474 (2022)) severely masking sugar/nutrient-evoked responses.

Calcium imaging data collection and analysis. Imaging data was obtained using an Evolve 512 EMCCD camera (Photometrics). Data was acquired at 5 Hz. A single field of view was chosen for recording and analysis from each ganglion. Calcium imaging data collected at 5 Hz was down-sampled by a factor of 2, and the images were stabilized using the NORMCorre algorithm. Motion-corrected movies were then manually segmented in ImageJ using the Cell Magic Wand plugin. Neuropil fluorescence was subtracted from each region of interest with the FISSA toolbox, and neural activity was denoised using the OASIS deconvolution algorithm. Neuronal activity was analyzed for significant stimulus-evoked responses as described previously ((Tan, H. E., et al., The gut-brain axis mediates sugar preference. Nature 580, 511-516 (2020); Barretto, R. P., et al., The neural representation of taste quality at the periphery. Nature 517, 373-376 (2015)). Note that for the fat receptor knockout imaging studies, the minimal peak amplitude for defining responders was set to 1% ΔF/F. To quantify responses in fat receptors knockouts (FIG. 6C, FIG. 16G), the number of responding neurons over the total number of imaged neurons per ganglia was normalized to the number of responders in wild-type control mice (set to 100%). For experiments using blockers, two repeat trials per stimuli were used to accommodate the expanded time scale of the session (that is, before and after), and a neuron was considered a responder if it responded in both trials. The two-trial average area under curve for each stimulus was used to quantify the before and after responses (FIG. 12D, FIG. 12E). Imaging data is presented as heat maps of z-score-normalized responses. Equivalent results are obtained when using absolute ΔF/F (data not shown).

Statistics. No statistical methods were used to predetermine sample size, and investigators were not blinded to group allocation. No method of randomization was used to determine how mice were allocated to experimental groups. Statistical methods used include one-way ANOVA followed by Tukey's honest significant difference (HSD) post hoc test, two-tailed t-test, two-way ANOVA or the two-sided Mann-Whitney U-test, and are indicated for all figures. Analyses were performed in MATLAB and GraphPad Prism 8. Data are presented as mean±s.e.m. FIG. 6C: ANOVA with Tukey's test compared to Snap25-GCaMP6s control. CD36 KO (n=6 mice) vs control, P=0.99; GPR40 KO (n=7 mice) vs control, P=0.89; GPR120 KO (n=6 mice) vs control, P=0.53; CD36/GPR40 double KO (n=6 mice) vs control, P=0.96; CD36/GPR120 double KO, (n=8 mice) vs control, P=0.99; GPR40/GPR120 double KO (n=7 mice) vs control, P=5×10⁻⁶; CD36/GPR40/GPR120 triple KO (n=6 mice) vs control, P=4×10⁻⁶. FIG. 6E: Two-tailed paired t-tests evaluating pre versus post fat preference. Wild-type mice (n=11 mice) pre vs post, P=2×10⁻⁶; CD36 KO (n=8 mice) pre vs post, P=4.8×10⁻³; GPR40 KO (n=12 mice) pre vs post, P=1×10⁻⁴; GPR120 KO (n=14 mice) pre vs post, P=1.03×10⁻⁴; CD36/GPR40 KO (n=5 mice) pre vs post, P=2×10⁻²; CD36/GPR120 KO (n=6 mice) pre vs post, P=1.7×10⁻³; GPR40/GPR 120 double KO (n=7 mice) pre vs post, P=0.81; CD36/GPR40/GPR120 triple KO (n=9 mice) pre vs post, P=0.46. FIG. 6F: Two-tailed paired t-tests evaluating pre versus post sugar preference. Wild-type mice (n=10 mice) pre vs post, P=2.9×10⁻⁵; Gpr40−/−Gpr120−/− (n=9 mice), pre vs post, P=8.0×10⁻⁵; Cd36−/−Gpr40−/−Gpr120−/− (n=7 mice), pre vs post, P=1.9×10⁻³.

The disclosures of each patent, patent application, and publication cited or described herein are hereby incorporated herein by reference, in its entirety.

Those skilled in the art will appreciate that numerous changes and modifications can be made to the preferred embodiments and that such changes and modifications can be made without departing from the spirit of the disclosed methods. It is, therefore, intended that the appended claims cover all such equivalent variations as fall within the true spirit and scope of the claimed methods.

### EMBODIMENTS

The following list of embodiments is intended to complement, rather than displace or supersede, the previous descriptions.

Embodiment 1. A method of regulating a subject's preference for fat and/or sugar comprising administering to the subject a molecule that activates or inhibits one or more of a GPR40 receptor, a GPR120 receptor, and a CCK signaling pathway.

Embodiment 2. The method of embodiment 1, wherein the molecule activates the GPR40 receptor and/or the GPR120 receptor.

Embodiment 3. The method of embodiment 1, wherein the molecule inhibits the GPR40 receptor and/or the GPR120 receptor.

Embodiment 4. The method of any one of the previous embodiments, wherein the molecule can activate or inhibit the downstream signaling pathway of GPR40 and/or GPR120.

Embodiment 5. The method of embodiment 4, wherein the molecule that activates or inhibits GPR40 and/or GPR120 or the downstream signaling pathway of GPR40 and/or GPR120 increases or decreases the subject's preference for fat.

Embodiment 6. The method of embodiment 1, wherein CCK signaling is activated or inhibited with a CCK peptide, Devazepide, a small molecule, or a peptide.

Embodiment 7. The method of any one of the previous embodiments, wherein activation of vagal sensory neurons is increased or decreased.

Embodiment 8. The method of embodiment 7, wherein the vagal sensory neurons express TRPA1, CCKAR, Uts2b, or vasoactive intestinal peptide (VIP).

Embodiment 9. A method of screening a substance for an ability to activate or reduce activation of a nutrient receptor in a subject's gut, the method comprising:
administering a substance to a subject wherein the subject's vagal sensory neurons express a genetically encoded calcium indicator (GECI);
imaging the vagal sensory neurons of the subject; and
detecting a presence or absence of a signal from the GECI, wherein the presence of the signal in a region of the subject's vagal sensory neurons that are responsive to nutrient receptor activation in the gut indicates that the substance activates the nutrient receptor, or wherein a reduction of the signal in a region of the subject's vagal sensory neurons that are otherwise responsive to nutrient receptor activation in the gut indicates that the substance inhibits the nutrient receptor.

Embodiment 10. The method of embodiment 9, wherein the substance is administered orally or by intestinal infusion.

Embodiment 11. The method of embodiment 9 or 10, wherein the substance comprises a fat, a sugar, an amino acid, or a combination thereof.

Embodiment 12. The method of embodiment 9 or 10, wherein the substance comprises a noncaloric compound.

Embodiment 13. The method of any one of embodiments 9-12, wherein the substance activates or reduces activation of GPR40 and/or GPR120.

Embodiment 14. The method of any one of embodiments 9-12, wherein the substance activates or reduces activation of CCK signaling pathway and/or TRPA1 vagal neuron pathway.

Embodiment 15. The method of any one of embodiments 9-14, wherein the neurons express TRPA1, CCKAR, Uts2b, or vasoactive intestinal peptide (VIP).

1. A method of regulating a subject's preference for fat and/or sugar, the method comprising administering to the subject a molecule that activates or inhibits one or more of a GPR40 receptor, a GPR120 receptor, and a CCK signaling pathway.

2. The method of claim 1, wherein the molecule activates the GPR40 receptor and/or the GPR120 receptor.

3. The method of claim 1, wherein the molecule inhibits the GPR40 receptor and/or the GPR120 receptor.

4. The method of claim 1, wherein the molecule can activate or inhibit the downstream signaling pathway of GPR40 and/or GPR120.

5. The method of claim 4, wherein the molecule that activates or inhibits GPR40 and/or GPR120 or the downstream signaling pathway of GPR40 and/or GPR 120 increases or decreases the subject's preference for fat.

6. The method of claim 1, wherein CCK signaling is activated or inhibited with a CCK peptide, Devazepide, a small molecule, or a peptide.

7. The method of any one of the previous claims, wherein activation of vagal sensory neurons is increased or decreased.

8. The method of claim 7, wherein the vagal sensory neurons express TRPA1, CCKAR, Uts2b, or vasoactive intestinal peptide (VIP).

9. A method of screening a substance for an ability to activate or reduce activation of a nutrient receptor in a subject's gut, the method comprising:
administering a substance to a subject wherein the subject's vagal sensory neurons express a genetically encoded calcium indicator (GECI);
imaging the vagal sensory neurons of the subject; and
detecting a presence or absence of a signal from the GECI, wherein the presence of the signal in a region of the subject's vagal sensory neurons that are responsive to nutrient receptor activation in the gut indicates that the substance activates the nutrient receptor, or wherein a reduction of the signal in a region of the subject's vagal sensory neurons that are otherwise responsive to nutrient receptor activation in the gut indicates that the substance inhibits the nutrient receptor.

10. The method of claim 9, wherein the substance is administered orally or by intestinal infusion.

11. The method of claim 9, wherein the substance comprises a fat, a sugar, an amino acid, or a combination thereof.

12. The method of claim 9, wherein the substance comprises a noncaloric compound.

13. The method of claim 9, wherein the substance activates or reduces activation of GPR40 and/or GPR120.

14. The method of claim 9, wherein the substance activates or reduces activation of CCK signaling pathway and/or TRPA1 vagal neuron pathway.

15. The method of claim 9, wherein the neurons express TRPA1, CCKAR, Uts2b, or vasoactive intestinal peptide (VIP).
